(19) United States
(12) Patent Application Publication
(10) Pub. No.: US 2022/0280928 A1
(43) Pub. Date: Sep. 8, 2022

(54) HETEROGENEOUS CATALYSTS, AND USES THEREOF

(71) Applicant: Novomer, Inc., Rochester, NY (US)

(72) Inventors: Derek Williams, Rochester, NY (US); Jonathan D. Tedder, Rochester, NY (US)

(21) Appl. No.: 17/630,181

(22) PCT Filed: Jul. 29, 2020
(86) PCT No.: PCT/US2020/044013

§ 371 Date: Jan. 26, 2022

Related U.S. Application Data
(60) Provisional application No. 62/882,059, filed on Aug. 2, 2019.

(51) Int. Cl. B01J 35/00; B01J 31/02; B01J 31/18; B01J 23/75

(52) U.S. Cl. CPC B01J 35/0006; B01J 31/0295; B01J 31/1815; B01J 23/75; B01J 2523/845; B01J 2531/0213; B01J 2540/66

(57) ABSTRACT

Provided herein are heterogeneous catalysts suitable for use in carbonylation reactions, including the production of acrylic acid from ethylene oxide and carbon monoxide on an industrial scale. The production may involve various unit operations, including, for example: a beta-propiolactone production system configured to produce beta-propiolactone from ethylene oxide and carbon monoxide; a polypropiolactone production system configured to produce polypropiolactone from beta-propiolactone; and an acrylic acid production system configured to produce acrylic acid with a high purity by thermolysis of polypropiolactone.

### FIELD

The present disclosure relates generally to systems and methods for producing beta-lactones from carbonylation of epoxides, and more specifically to the use of heterogeneous catalysts in such systems and methods. The beta-lactones, such as beta-propiolactone, may be used to produce polypropiolactone and acrylic acid.

### BACKGROUND

Polypropiolactone is a biodegradable polymer that can be used in many packaging and thermoplastic applications. Polypropiolactone is also a useful precursor for the production of acrylic acid. Polypropiolactone may serve as a precursor for acrylic acid, which is in high demand for the production of polyacrylic-acid-based superabsorbent polymers, detergent co-builders, dispersants, flocculants and thickeners. One advantage of polypropiolactone is that it can be safely transported and stored for extended periods of time without the safety or quality concerns associated with shipping and storing acrylic acid. There additionally is interest in acrylic acid that can be produced from biomass-derived feedstock, petroleum-derived feedstock, or combinations thereof. Given the size of the acrylic acid market and the importance of downstream applications of acrylic acid, there is a need for industrial systems and methods to produce acrylic acid and precursors thereof.

### BRIEF SUMMARY

Provided herein are methods and systems for producing beta-lactone products from carbonylating epoxides in the presence of heterogeneous catalysts. These beta-lactone products, such as beta-propiolactone, may be converted into useful downstream products, such as acrylic acid.

In some aspects, provided is a heterogeneous catalyst, comprising: a solid support; at least one ligand coordinated to a metal atom to form a metal complex; at least one anionic metal carbonyl moiety coordinated to the metal complex; and at least one linker moiety connecting each ligand to the solid support.

In some embodiments, the at least one ligand is a porphyrin ligand or a salen ligand. In some embodiments, the at least one anionic metal carbonyl moiety is a cobalt carbonyl moiety. In some embodiments, the solid support comprises silica, magnesia, alumina, titania, zirconia, zincate, carbon, or zeolite, or any combination thereof. In some embodiments, the at least one linker moiety comprises a sulfonate moiety or an aminosiloxane moiety.

In other aspects, provided is a heterogeneous catalyst, comprising: a solid support comprising a plurality of pores; at least one ligand coordinated to a metal atom to form a metal complex, wherein each ligand is encapsulated within the pores of the solid support; and at least one anionic metal carbonyl moiety coordinated to the metal complex. In some embodiments, the solid support is zeolite.

In yet other aspects, provided is a method, comprising reacting an epoxide with carbon monoxide in the presence of a heterogeneous catalyst, as described herein, to produce a beta-lactone product. In some embodiments, provided is a method, comprising: reacting an epoxide with carbon monoxide in the presence of a heterogeneous catalyst, as described herein, and a solvent to produce a product stream, which comprises a beta-lactone product and the solvent; and purifying the product stream by distillation to separate the product stream into a solvent recycle stream and a purified beta-lactone stream. The solvent recycle stream comprises the solvent, and the purified beta-lactone stream comprises the beta-lactone product.

In certain aspects, provided is also a system, comprising: a beta-lactone production system and a beta-lactone purification system. In some embodiments, the beta-lactone production system comprises: a carbon monoxide source; an epoxide source; a solvent source; a carbonylation reactor, such as a fixed or fluid bed reactor, that contains a catalyst comprising any of the heterogeneous catalysts described herein. The reactor also has at least one inlet to receive carbon monoxide from the carbon monoxide source, epoxide from the epoxide source, and solvent from the solvent source, and an outlet to output a beta-lactone stream, wherein the beta-lactone stream comprises a beta-lactone product and solvent. In some embodiments, the beta-lactone purification system comprises: at least one distillation column configured to receive the beta-lactone stream from the carbonylation reactor, and to separate the beta-lactone stream into a solvent recycle stream and a purified beta-lactone stream.

In one variation of the method and system described above and herein, the epoxide is ethylene oxide, and the beta-lactone product is beta-propiolactone.

### DESCRIPTION OF THE FIGURES

The present application can be best understood by reference to the following description taken in conjunction with the accompanying figures, in which like parts may be referred to by like numerals.

FIG. 1 depicts one exemplary general reaction scheme to produce acrylic acid from ethylene oxide and carbon monoxide.

FIG. 2 is a schematic illustration of a system to produce acrylic acid from carbon monoxide and ethylene oxide.

FIG. 3 is a schematic illustration of the unit operations to produce polypropiolactone from beta-propiolactone, and acrylic acid from polypropiolactone.

FIG. 4 is a schematic illustration of a system for converting beta-propiolactone to polypropiolactone that involves the use of two continuous stirred-tank reactors in series.

FIG. 5 is a schematic illustration of a system for converting beta-propiolactone to polypropiolactone that involves the use of two loop reactors in series.

FIG. 6 is a schematic illustration of a system for converting beta-propiolactone to polypropiolactone that involves a plug flow reactor with multiple cooling zones.

FIGS. 7-14 depict various configurations of production systems to produce acrylic acid from ethylene oxide and carbon monoxide, via the production of beta-propiolactone and polypropiolactone.

FIG. 15 illustrates an embodiment of an acrylic acid production system described herein.

FIG. 16 illustrates an embodiment of a carbonylation reaction system described herein.

FIG. 17 illustrates an embodiment of a BPL purification system described herein.

FIGS. 18A-18D depict a series of reactions described in Example 1 to produce an exemplary heterogeneous catalyst, compound (5) (FIG. 18D).

FIGS. 19A-19C depict a series of reactions described in Example 2 to produce another exemplary heterogeneous catalyst, compound (4) (FIG. 19C).

FIG. 20 depicts an exemplary heterogeneous catalyst as described in Example 3, in which a salen ligand is encapsulated in one of the pores of the solid support.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

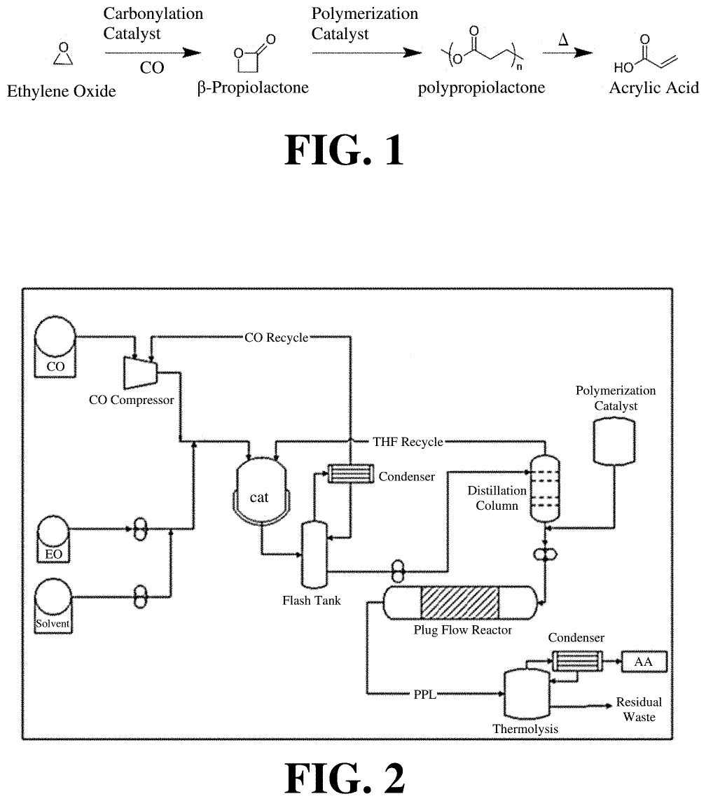

Organic acids, such as acrylic acid, may be produced by conversion of a beta-lactone and/or thermal decomposition of a polylactone comprising beta-lactone monomers. Such beta-lactone may be produced by carbonylation of an epoxide (e.g., in the presence of carbon monoxide). For example, in one aspect, acrylic acid can be produced from ethylene oxide and carbon monoxide according to the following exemplary general reaction scheme depicted in FIG. 1. Ethylene oxide (“EO”) may undergo a carbonylation reaction, e.g., with carbon monoxide (“CO”), in the presence of a carbonylation catalyst to produce beta-propiolactone (“BPL”). The beta-propiolactone may undergo polymerization in the presence of a polymerization catalyst to produce polypropiolactone (“PPL”). The polypropiolactone may undergo thermolysis to produce acrylic acid (“AA”).

With respect to the carbonylation of epoxides, provided herein are heterogeneous carbonylation catalysts. In some aspects, provided is a method comprising reacting an epoxide with carbon monoxide in the presence of such heterogeneous catalyst to produce a beta-lactone.

Such heterogeneous catalysts may be used in a fixed or fluid bed reactor to produce the BPL. The resulting BPL product stream generally does not need to be further purified to separate residual carbonylation catalyst, and the catalyst consumption is generally lower than when homogeneous catalysts are used. For example, when a homogeneous carbonylation catalyst is used, the BPL product stream may undergo nanofiltration to separate residual carbonylation catalyst present, and such separated carbonylation catalyst may be recycled for use in the carbonylation reactor. Such nanofiltration step can be avoided when the heterogeneous catalysts described herein are used as the carbonylation catalysts.

Thus, in other aspects, provided is a method comprising reacting an epoxide with carbon monoxide in the presence of a catalyst and a solvent to produce a product stream. The catalyst comprises any of the heterogeneous catalysts described herein. The product stream comprises BPL and the solvent. The method further comprises purifying such product stream by distillation to separate the product stream into a solvent recycle stream and a purified BPL stream.

The heterogeneous catalysts, methods of making them, as well as methods of using them are described in further detail below.

### Heterogeneous Catalysts

In some aspects, provided are heterogeneous catalysts suitable for use in the carbonylation of epoxides.

In some embodiments, provided is a heterogeneous catalyst, comprising: a solid support; at least one ligand coordinated to a metal atom to form a metal complex; at least one anionic metal carbonyl moiety coordinated to the metal complex; and at least one linker moiety connecting each ligand to the solid support.

Solid Support

In some variations, the solid support comprises silica, magnesia, alumina, titania, zirconia, zincate, carbon, or zeolite.

In certain variations, the solid support comprises silica. In one variation, the solid support comprises silica/alumina, pyrogenic silica, or high purity silica.

In some embodiments, the solid support is porous. In some embodiments, the solid support comprises a plurality of pores.

In certain embodiments, the solid support comprises zeolite.

In some variations, the solid support comprises zeolite having pore dimensions smaller than about 10 Å. In certain variations, zeolite materials that can be used as suitable solid supports include certain small pore faujasites, medium pore pentasils, small pore ferrierite, two-dimensional large pore mordenite, large pore β-type materials and basic zeolites. In certain variations, the solid support comprises basic zeolites. In one variation, the solid support comprises X or Y zeolites. In another variation, the solid support comprises X zeolite or Y zeolite in sodium form (NaX or NaY), zeolite L in potassium form (KL), or synthetic ferrierite. In another variations, the solid support comprises medium pore, pentasil-type zeolite having 10-membered oxygen ring systems, such as ZSM-5, ZSM-11, ZSM-22, ZSM-23, ZSM-48 and laumontite. In other variations, the solid support comprises zeolite having dual pore systems displaying interconnecting channels of either 12- and 8-membered oxygen ring openings or 10- and 8-membered oxygen ring openings. In certain variations, the solid support comprises mordenite, offretite, Linde T, gmelinite, heulandite/clinoptilolite, ferrierite, ZSM-35, ZSM-38, stilbite, dachiardite, or epistilbite. In one variation, the solid support comprises zeolite having dual pore systems and/or pore dimensions of about 3 to 8 Å. In another variation, the solid support comprises pentasil ZSM-5, ferrierite, mordenite, or Y-zeolite in sodium form (NaY) in addition to alumina, silica/alumina and zeolite alumina.

Any combinations of solid supports described herein may also be used.

Linker Moieties

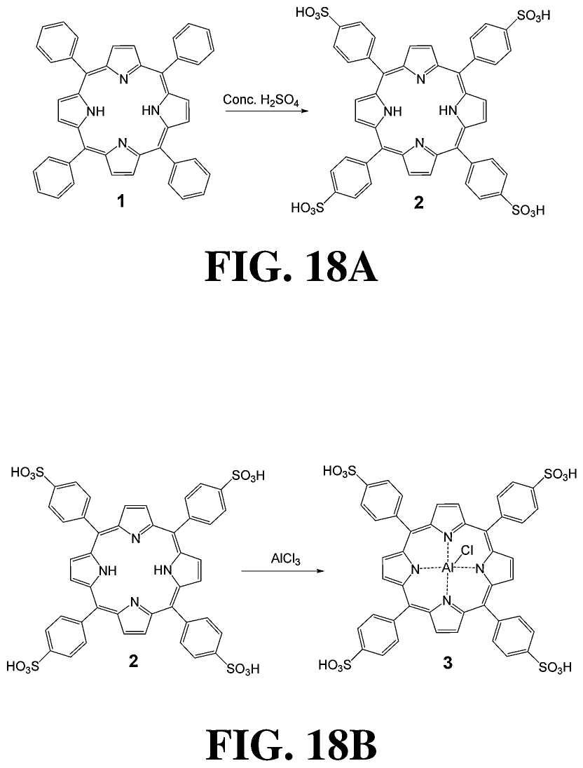

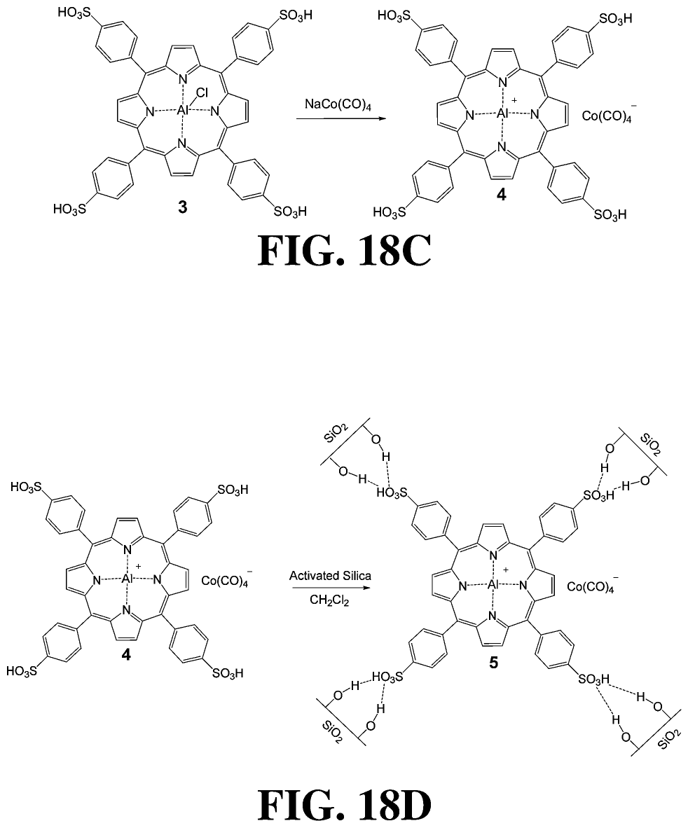

In some variations, the linker moiety comprises a sulfonate moiety. In one variation, the linker moiety comprises —SO₃H—. For example, as depicted in FIG. 18D, the —SO₃H— moiety links the metal complex to the —OH groups in the silica support.

In other variations, the linker moiety comprises an aminosiloxane moiety. In certain variations, the linker moiety comprises a moiety of formula (LM1):

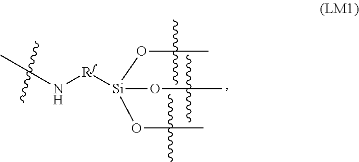

wherein R^{f} is an optionally substituted -alkyl- moiety.

In certain embodiments, the -alkyl- moiety includes —(CH₂)ₙ—, wherein n is an integer greater than 0. In one variation, n is 1-10, or 1-5, or 1, 2, 3, 4, or 5. In one embodiment, the -alkyl- moiety is, for example, —CH₂—, —CH₂CH₂—, or —CH₂CH₂CH₂—.

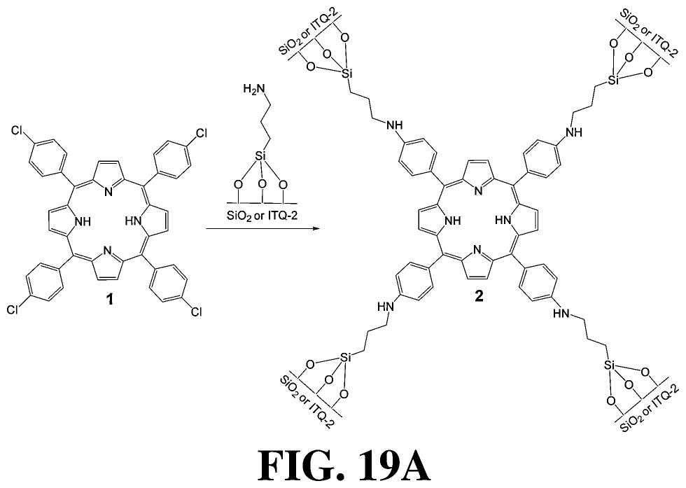

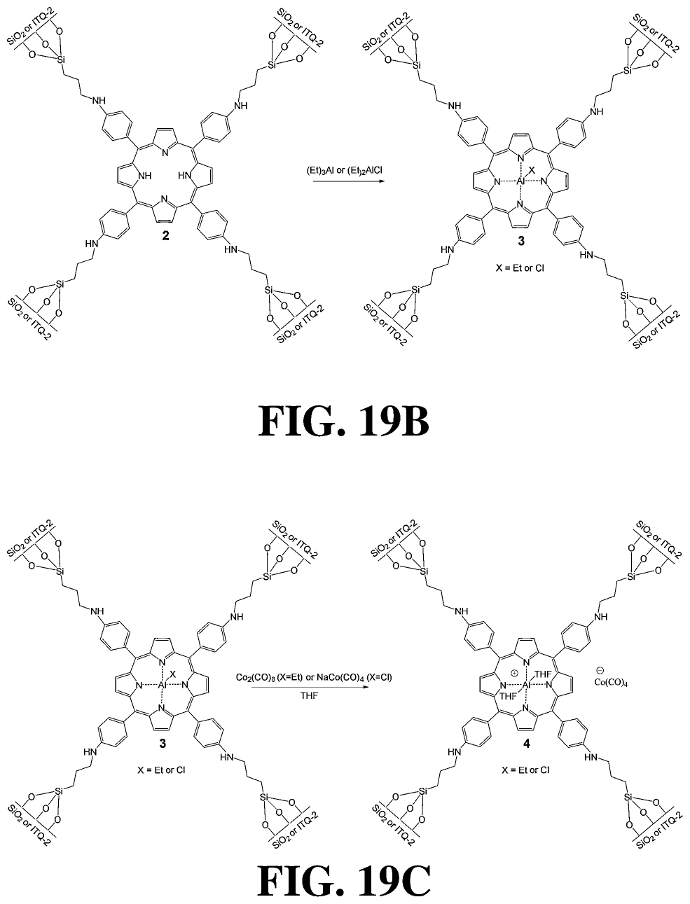

For example, as depicted in FIG. 19C, the

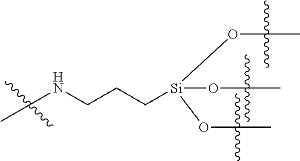 [SMILES: *NCCC[Si](O*)(O*)O*]

moiety links the metal complex to the silica or titania support.

Any combinations of linker moieties described herein may also be used.

Further, in certain embodiments, the metal complex is linked to the solid support by one or more linker moieties. In one embodiment, the metal complex is linked to the solid support by a plurality of linker moieties.

Ligands

In some variations, the ligand is a porphyrin ligand or a salen ligand.

In certain variations, the ligand is a ligand of formula (L-A):

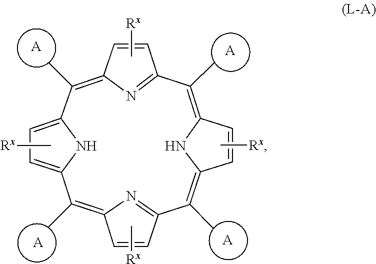

wherein:
each R^{x} is independently H or a substituent as defined below; and
each ring A is independently optionally substituted (as defined below), and wherein at least one ring A is connected by the linker moiety to the solid support.

In certain variations, the ligand is a ligand of formula (L-A1):

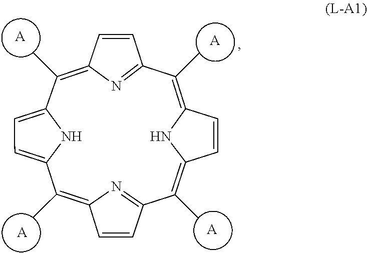

wherein each ring A is independently optionally substituted, and wherein at least one ring A is connected by the linker moiety to the solid support.

In some variations of the foregoing, at least one, at least two, or at least three, or one, two, three or four rings A are connected by linker moieties to the solid support.

In some variations of the foregoing, each ring A is independently a 6-membered cyclic moiety. In certain variations, each ring A is independently a carbocyclic moiety or a heterocyclic moiety. In one variation, the heterocyclic moiety comprises at least one nitrogen atom.

In one variation of the foregoing, the ligand is a ligand of formula (L-A2):

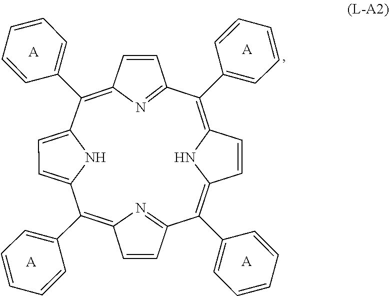

wherein each ring A is independently optionally substituted, and wherein at least one ring A is connected by the linker moiety to the solid support.

In certain variations of the foregoing, each ring A is connected in the para position by the linker moiety to the solid support.

In another variation, the ligand is a ligand of formula (L-A3):

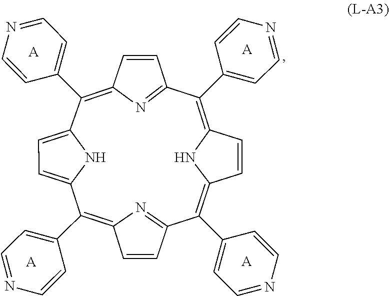

wherein each ring A is independently optionally substituted, and wherein at least one ring A is connected by the linker moiety to the solid support.

In certain variations, the ligand is a ligand of formula (L-B):

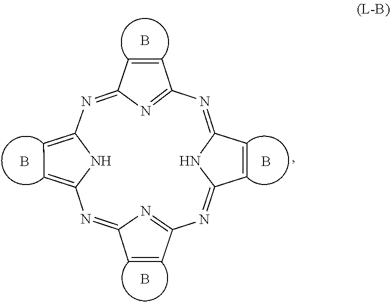

wherein each ring B is independently optionally substituted, and wherein at least one ring B is connected by the linker moiety to the solid support.

In some variations of the foregoing, at least one, at least two, or at least three, or one, two, three or four rings B are connected by linker moieties to the solid support.

In some variations, each ring B is independently a 6-membered cyclic moiety. In certain variations, each ring B is independently a carbocyclic moiety or a heterocyclic moiety. In one variation, the heterocyclic moiety comprises at least one nitrogen atom.

In one variation of the foregoing, the ligand is a ligand of formula (L-B1):

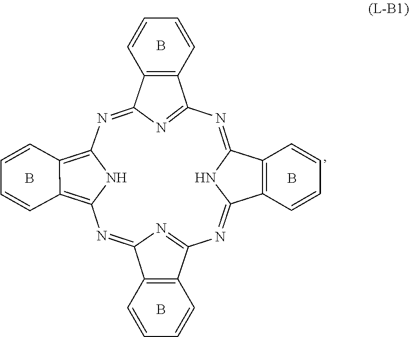

wherein each ring B is independently optionally substituted, and wherein at least one ring B is connected by the linker moiety to the solid support.

In certain variations, the ligand is a ligand of formula (L-C):

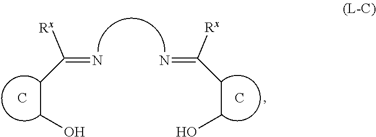

wherein:
each R^{x} is independently H or a substituent as defined below;
[symbol] is an optionally substituted moiety linking the two nitrogen atoms of the diamine portion of the ligand; and
each ring C is independently optionally substituted, and wherein at least one ring C is connected by the linker moiety to the solid support.

In certain variations, the ligand is a ligand of formula (L-C1):

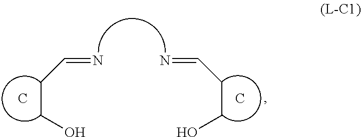

wherein:
[symbol] is an optionally substituted moiety linking the two nitrogen atoms of the diamine portion of the salen ligand, and
each ring C is independently optionally substituted, and wherein at least one ring C is connected by the linker moiety to the solid support.

In some variations of the foregoing, one or both rings C are connected by linker moieties to the solid support.

In some variations of the foregoing, each ring C is independently a 6-membered cyclic moiety. In certain variations, each ring C is independently a carbocyclic moiety or a heterocyclic moiety. In one variation, the heterocyclic moiety comprises at least one nitrogen atom.

In some variations of the foregoing, [symbol] is an optionally substituted C₃-C₁₄ carbocycle, a C₆-C₁₀ aryl group, a C₃-C₁₄ heterocycle, a C₅-C₁₀ heteroaryl group, or a C₂₋₂₀ aliphatic group.

In one variation, the ligand is a ligand of formula (L-C2):

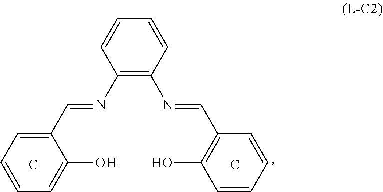

wherein each ring C is independently optionally substituted, and wherein at least one ring C is connected by the linker moiety to the solid support.

In another variation, the ligand is a ligand of formula (L-C3):

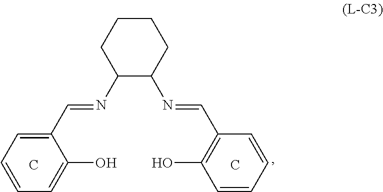

wherein each ring C is independently optionally substituted, and wherein at least one ring C is connected by the linker moiety to the solid support.

In some variations, the ligand is a ligand of formula (L-D):

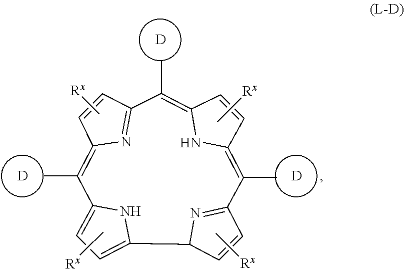

wherein:
each R^{x} is independently H or a substituent as defined below;
each ring D is independently optionally substituted, and wherein at least one ring D is connected by the linker moiety to the solid support.

In some variations of the foregoing, at least one, or at least two, or one, two, or three rings D are connected by linker moieties to the solid support.

In some variations, each ring D is independently a 6-membered cyclic moiety. In certain variations, each ring D is independently a carbocyclic moiety or a heterocyclic moiety. In one variation, the heterocyclic moiety comprises at least one nitrogen atom.

In one variation of the foregoing, the ligand is a ligand of formula (L-D1):

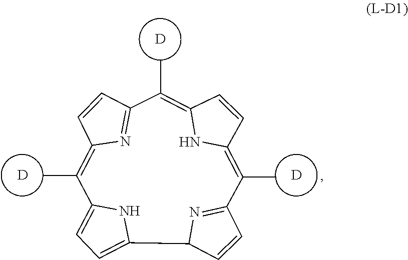

wherein each ring D is independently optionally substituted, and wherein at least one ring D is connected by the linker moiety to the solid support.

In another variation of the foregoing, the ligand is a ligand of formula (L-D2):

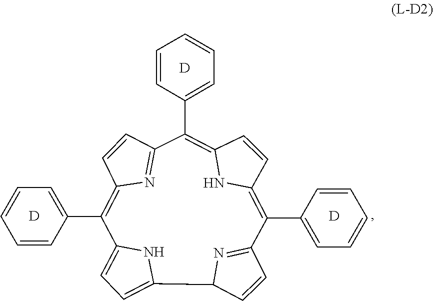

wherein each ring D is independently optionally substituted, and wherein at least one ring D is connected by the linker moiety to the solid support.

In certain variations of the foregoing, at least one ring D is connected in the para position by the linker moiety to the solid support.

In other variations, the ligand is a ligand of formula (L-E):

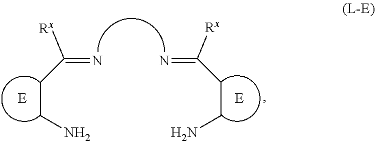

wherein:
each R^{x} is independently H or a substituent as defined below;
[symbol] is an optionally substituted moiety linking the two nitrogen atoms of the diamine portion of the ligand; and
each ring E is independently optionally substituted, and wherein at least one ring E is connected by the linker moiety to the solid support.

In some variations of the foregoing, one or both rings E are connected by linker moieties to the solid support.

In some variations, each ring E is independently a 6-membered cyclic moiety. In certain variations, each ring E is independently a carbocyclic moiety or a heterocyclic moiety. In one variation, the heterocyclic moiety comprises at least one nitrogen atom.

In another variation, the ligand is a ligand of formula (L-E1):

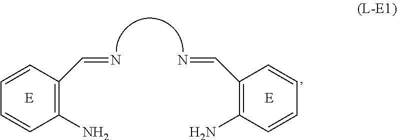

wherein:
[symbol] is an optionally substituted moiety linking the two nitrogen atoms of the diamine portion of the ligand; and
each ring E is independently optionally substituted, and wherein at least one ring E is connected by the linker moiety to the solid support.

In some variations of the foregoing, [symbol] is an optionally substituted C₃-C₁₄ carbocycle, a C₆-C₁₀ aryl group, a C₃-C₁₄ heterocycle, a C₅-C₁₀ heteroaryl group, or a C₂₋₂₀ aliphatic group.

In one variation, the ligand is a ligand of formula (L-E2):

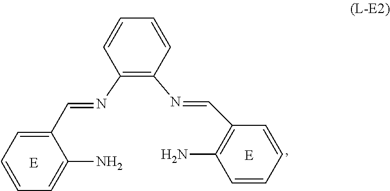 [SMILES: Nc1ccccc1/C=N/c1ccccc1/N=C/c1ccccc1N]

wherein each ring E is independently optionally substituted, and wherein at least one ring E is connected by the linker moiety to the solid support.

In another variation, the ligand is a ligand of formula (L-E3):

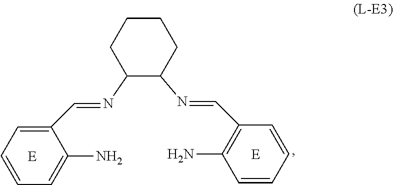 [SMILES: Nc1ccccc1/C=N/C1CCCCC1/N=C/c1ccccc1N]

wherein each ring E is independently optionally substituted, and wherein at least one ring E is connected by the linker moiety to the solid support.

In yet other variations, the ligand is a ligand of formula (L-F):

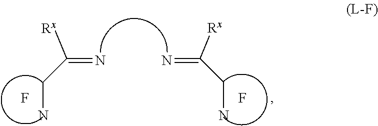

wherein:
each R^{x} is independently H or a substituent as defined below;
[symbol] is an optionally substituted moiety linking the two nitrogen atoms of the diamine portion of the ligand; and
each ring F is independently optionally substituted, and wherein at least one ring F is connected by the linker moiety to the solid support.

In one variation, the ligand is a ligand of formula (L-F1):

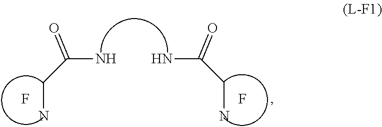

wherein:
[symbol] is an optionally substituted moiety linking the two nitrogen atoms of the diamine portion of the ligand, and
each ring F is independently optionally substituted, and wherein at least one ring F is connected by the linker moiety to the solid support.

In some variations of the foregoing, one or both rings F are connected by linker moieties to the solid support.

In some variations of the foregoing, each ring F is independently a 6-membered heterocyclic moiety. In one variation, the heterocyclic moiety comprises at least two nitrogen atoms. In certain variations, each ring F is independently a heteroaryl.

In another variation, the ligand is a ligand of formula (L-F2):

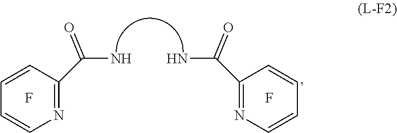

wherein:
[symbol] is an optionally substituted moiety linking the two nitrogen atoms of the diamine portion of the ligand, and
each ring F is independently optionally substituted, and wherein at least one ring F is connected by the linker moiety to the solid support.

In some variations of the foregoing, [symbol] is an optionally substituted C₃-C₁₄ carbocycle, a C₆-C₁₀ aryl group, a C₃-C₁₄ heterocycle, a C₅-C₁₀ heteroaryl group, or a C₂₋₂₀ aliphatic group.

In yet another variation, the ligand is a ligand of formula (L-F3):

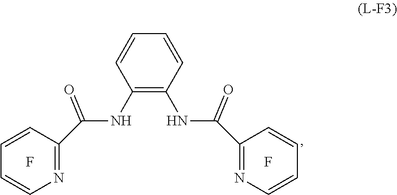

wherein each ring F is independently optionally substituted, and wherein at least one ring F is connected by the linker moiety to the solid support.

In yet another variation, the ligand is a ligand of formula (L-F4):

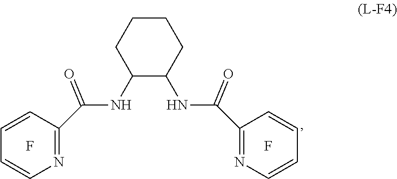

wherein each ring F is independently optionally substituted, and wherein at least one ring F is connected by the linker moiety to the solid support.

In yet other variations, the ligand is a ligand of formula (L-G):

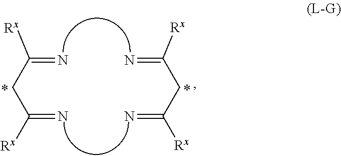

wherein:
each R^{x} is independently H or a substituent as defined below;
each [symbol] is independently an optionally substituted moiety linking the two nitrogen atoms of the diamine portion of the ligand; and
* is a position at which the atom at that position is connected by the linker moiety to the solid support, and wherein at least one of the two atoms at * are connected by the linker moiety to the solid support.

In certain variations, the ligand is a ligand of formula (L-G1):

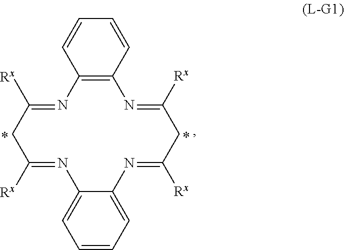

wherein:
each R^{x} is independently H or a substituent as defined below; and
* is a position at which the atom at that position is connected by the linker moiety to the solid support, and wherein at least one of the two atoms at * are connected by the linker moiety to the solid support.

In yet other variations, the ligand is a ligand of formula (L-G2):

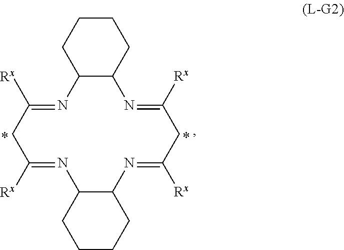

wherein:
each R^{x} is independently H or a substituent as defined below; and
* is a position at which the atom at that position is connected by the linker moiety to the solid support, and wherein at least one of the two atoms at * are connected to the solid support.

In some variations of the foregoing, the atom at one of the two * positions is connected to the solid support. In other variations, the atoms at both * positions are connected to the solid support.

In some embodiments, the substituents on rings A, B, C, D, E, and F, as well as the substituent of R^{x} may include: halo, —NO₂, —CN, —SR^{y}, —S(O)R^{y}, —S(O)₂R^{y}, —S(O)₂NR^{y}, —NR^{y}C(O)R^{y}, —OC(O)R^{y}, —CO₂R^{y}, —NCO, —CNO, —N₃, —SiR^{y}₃, —OR⁴, —OC(O)N(R^{y})₂, —N(R^{y})₂, —NR^{y}C(O)R^{y}, —NR^{y}C(O)OR^{y}. In other embodiments, the substituents on rings A, B, C, D, E, and F, and the substituent of R^{x}, may include: an optionally substituted C₁₋₂₀ aliphatic; an optionally substituted C₁₋₂₀ heteroaliphatic having 1-4 heteroatoms independently selected from nitrogen, oxygen, and sulfur; an optionally substituted 6- to 10-membered aryl; an optionally substituted 5- to 10-membered heteroaryl having 1-4 heteroatoms independently selected from nitrogen, oxygen, and sulfur; and an optionally substituted 4- to 7-membered heterocyclic having 1-2 heteroatoms independently selected from nitrogen, oxygen, and sulfur.

In some variations of the foregoing, each R^{y} is independently an optionally substituted C₁₋₆ aliphatic; an optionally substituted aryl; an optionally substituted 3-7 membered saturated or partially unsaturated carbocyclic ring; an optionally substituted 3-7 membered saturated or partially unsaturated monocyclic heterocyclic ring having 1-2 heteroatoms independently selected from nitrogen, oxygen, and sulfur; an optionally substituted 5-6 membered heteroaryl ring having 1-3 heteroatoms independently selected from nitrogen, oxygen, and sulfur; and an optionally substituted 8- to 10-membered aryl.

Metal Atom

In some variations, the metal atom is Ti, Cr, Mn, Fe, Ru, Co, Rh, Sm, Re, Jr, Zr, Ni, Pd, Zn, Mg, Al, Ga, Sn, In, Mo, or W. In certain variations, the metal atom is Zn, Cu, Mn, Co, Ru, Fe, Rh, Ni, Pd, Mg, Al, Cr, Ti, Fe, In, or Ga. In certain variations, the metal atom is Zn(II), Cu(II), Mn(II), Mn(III), Co(II), Co(III), Ru(II), Fe(II), Rh(II), Ni(II), Pd(II), Mg(II), Al(III), Cr(III), Cr(IV), Ti(III), Ti(IV), Fe(III), In(III), or Ga(III).

In one variation, the metal atom is aluminum. In another variation, the metal atom is chromium.

Metal Complex

The coordination of the ligand(s) to the metal atom forms the metal complex. Any of the ligands described herein, including a ligand of formula (L-A), (L-A1), (L-A2), (L-A3), (L-B), (L-B1), (L-C), (L-C1), (L-C2), (L-C3), (L-D), (L-D1), (L-D2), (L-E), (L-E1), (L-E2), (L-E3), (L-F), (L-F1), (L-F2), (L-F3), (L-F4), (L-G), (L-G1), or (L-G2) may coordinate with any of the metal atoms described herein, to the extent it is chemically feasible.

For example, in some variations, when the ligand of formula (L-A1) is used, the metal complex has a structure of formula (M-A1):

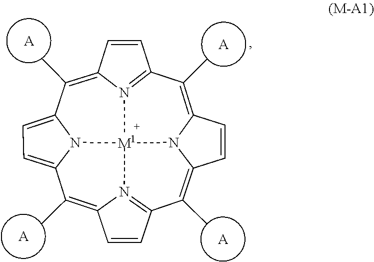

wherein:
M¹ is a metal atom; and
ring A is optionally substituted, and wherein each ring A is connected by the linker moiety to the solid support.

In other variations, when the ligand of formula (L-B) is used, the metal complex has a structure of formula (M-B):

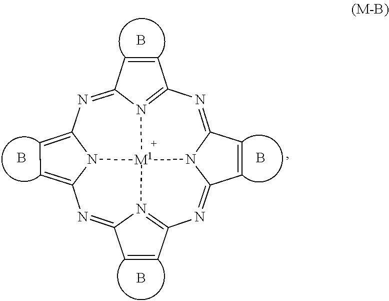

wherein:
M¹ is a metal atom; and
ring B is optionally substituted, and wherein each ring B is connected by the linker moiety to the solid support.

In other variations, when the ligand of formula (L-C1) is used, the metal complex has a structure of formula (M-C1):

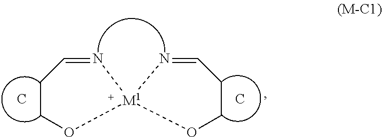

wherein:
M¹ is a metal atom;
[symbol] is an optionally substituted moiety linking the two nitrogen atoms of the diamine portion of the salen ligand; and
ring C is optionally substituted, and wherein each ring C is connected by the linker moiety to the solid support.

In other variations, when the ligand of formula (L-D1) is used, the metal complex has a structure of formula (M-D1):

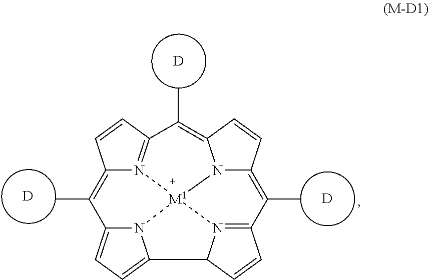

wherein:
M¹ is a metal atom; and
ring D is optionally substituted, and wherein each ring D is connected by the linker moiety to the solid support.

In other variations, when the ligand of formula (L-E1) is used, the metal complex has a structure of formula (M-E1):

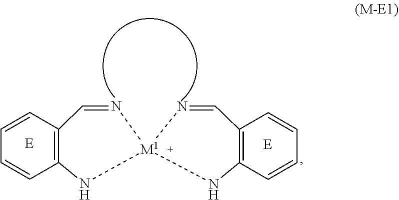

wherein:
M¹ is a metal atom,
[symbol] is an optionally substituted moiety linking the two nitrogen atoms of the diamine portion of the ligand, and
ring E is optionally substituted, and wherein each ring E is connected by the linker moiety to the solid support.

In yet other variations, when the ligand of formula (L-F2) is used, the metal complex has a structure of formula (M-F2):

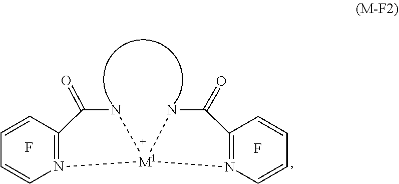

wherein:
M¹ is a metal atom,
[symbol] is an optionally substituted moiety linking the two nitrogen atoms of the diamine portion of the ligand, and
ring F is optionally substituted, and wherein each ring F is connected by the linker moiety to the solid support.

In still other variations, when the ligand of formula (L-G1) is used, the metal complex has a structure of formula (M-G1):

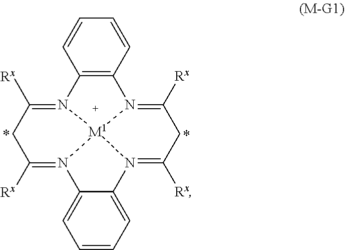

wherein:
each R^{x} is independently H or a substituent as defined below; and
* is a position at which the atom at that position is connected by the linker moiety to the solid support, and wherein at least one of the two atoms at * are connected by the linker moiety to the solid support.

It should be understood that any of the ligands of formulae (L-A), (L-A1), (L-A2), (L-A3), (L-B), (L-B1), (L-C), (L-C1), (L-C2), (L-C3), (L-D), (L-D1), (L-D2), (L-E), (L-E1), (L-E2), (L-E3), (L-F), (L-F1), (L-F2), (L-F3), (L-F4), (L-G), (L-G1), and (L-G2) may coordinate with a metal atom, M¹, to produce the corresponding metal complex of formulae (M-A), (M-A1), (M-A2), (M-A3), (M-B), (M-B1), (M-C), (M-C1), (M-C2), (M-C3), (M-D), (M-D1), (M-D2), (M-E), (M-E1), (M-E2), (M-E3), (M-F), (M-F1), (M-F2), (M-F3), (M-F4), (M-G), (M-G1) and (M-G2), respectively:

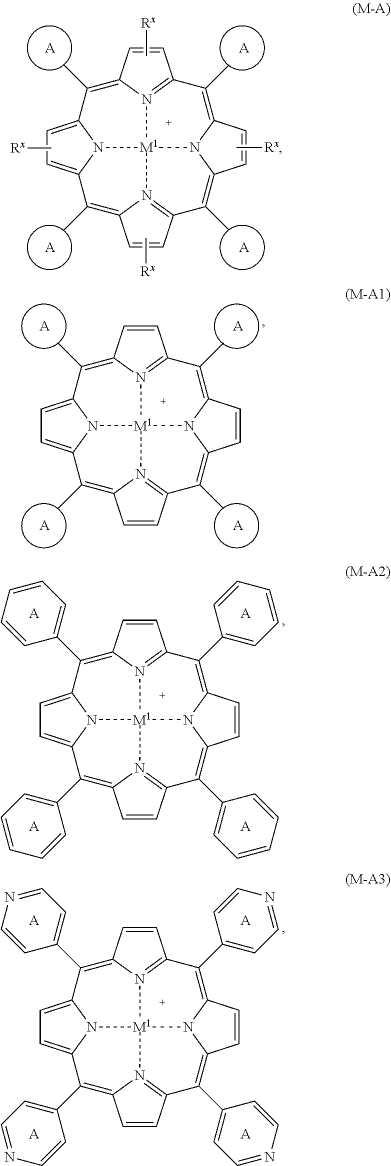

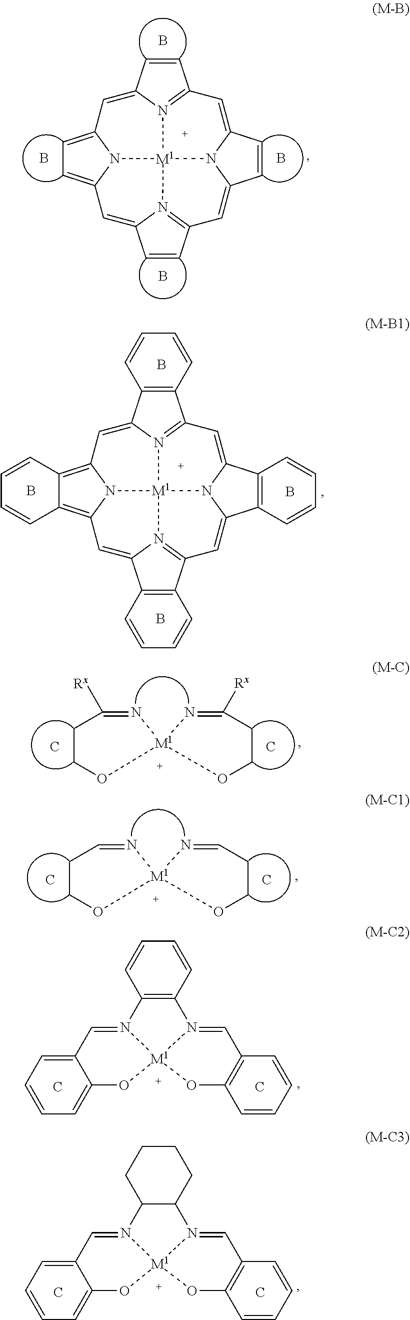

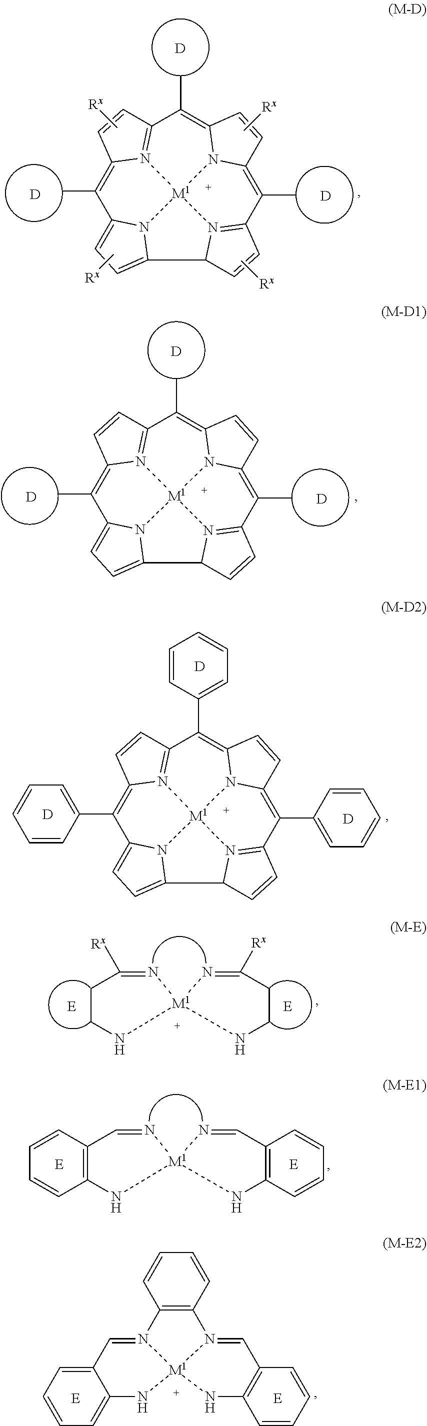

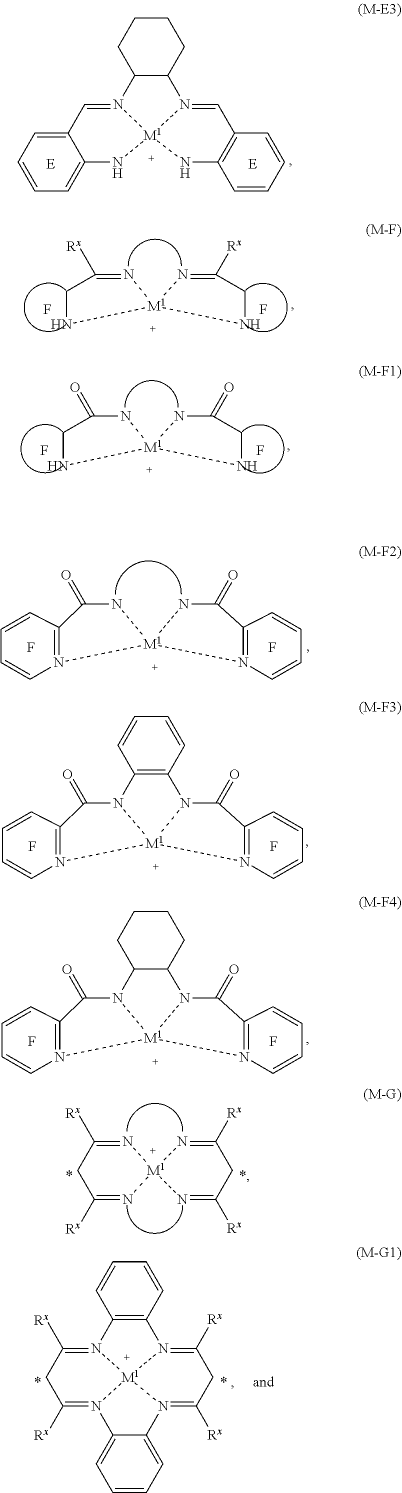

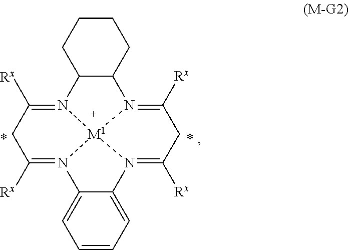

wherein the variables of each formula above are as defined herein.

It should be understood that any of the variations of the metal atoms, [symbol] , rings A-F, R^{x} and the optional substituents as described for the ligands also apply to their corresponding metal complexes.

Anionic Metal Carbonyl Moiety

In some embodiments, the anionic metal carbonyl moiety has the general formula [Q_{d}M′ₑ(CO)_{w}]^{y-}, where Q is a ligand and need not be present (if d is 0), M′ is a metal atom, d is an integer between 0 and 8 inclusive, e is an integer between 1 and 6 inclusive, w is a number such as to provide the stable anionic metal carbonyl moiety, and y is the charge of the anionic metal carbonyl moiety. In some variations, the anionic metal carbonyl moiety has the general formula [QM′(CO)_{w}]^{y-}, where Q is a ligand, M′ is a metal atom, w is a number such as to provide the stable anionic metal carbonyl moiety, and y is the charge of the anionic metal carbonyl moiety.

In some embodiments, the anionic metal carbonyl moiety includes monoanionic carbonyl complexes of metals from groups 5, 7 or 9 of the periodic table or dianionic carbonyl complexes of metals from groups 4 or 8 of the periodic table. In some embodiments, the anionic metal carbonyl compound contains cobalt or manganese. In some embodiments, the anionic metal carbonyl compound contains rhodium. Suitable anionic metal carbonyl compounds include, for example: [Co(CO)₄]⁻, [Ti(CO)₆]²⁻, [V(CO)₆]⁻ [Rh(CO)₄]⁻, [Fe(CO)₄]²⁻, [Fe₂(CO)₈]²⁻, [Ru(CO)₄]²⁻, [Os(CO)₄]²⁻, [Cr₂(CO)₁₀]²⁻, [Tc(CO)₅]⁻, [Re(CO)₅]⁻, and [Mn(CO)₅]⁻.

In some variations, the anionic metal carbonyl moiety is a cobalt carbonyl moiety. In one variation, the cobalt carbonyl moiety is [Co(CO)₄]⁻.

In some embodiments, a mixture of two or more anionic metal carbonyl complexes may be present in the heterogeneous catalysts used in the methods.

The term “such as to provide a stable anionic metal carbonyl moiety” for [Q_{d}M′ₑ(CO)_{w}]^{y-} is used herein to mean that [Q_{d}M′ₑ(CO)_{w}]^{y-} is a species characterizable by analytical means, e.g., NMR, IR, X-ray crystallography, Raman spectroscopy and/or electron spin resonance (EPR) and isolable in catalyst form in the presence of a suitable cation or a species formed in situ. It is to be understood that metals which can form stable metal carbonyl complexes have known coordinative capacities and propensities to form polynuclear complexes which, together with the number and character of optional ligands Q that may be present and the charge on the complex, will determine the number of sites available for carbon monoxide to coordinate and, therefore, the value of w. Typically, such compounds conform to the “18-electron rule”. Such knowledge is within the grasp of one having ordinary skill in the arts pertaining to the synthesis and characterization of metal carbonyl compounds.

### Methods of Producing the Heterogeneous Catalysts

In certain aspects, provided are also methods of producing the heterogeneous catalysts described herein. Various methods and techniques may be employed to produce such heterogeneous catalysts, including for example, adsorption, covalent tethering, and encapsulation.

Adsorption

In one aspect, provided is a method of producing a heterogeneous catalyst by: sulfonating at least one ligand to produce at least one sulfonated ligand; metallating the sulfonated ligand; reacting the metallated-sulfonated ligand with an anionic metal carbonyl moiety to produce a metal complex; and grafting the metal complex onto a solid support.

With reference to FIGS. 18A-D together, an exemplary reaction scheme is depicted to produce an exemplary heterogeneous catalyst according to such method. As depicted in catalyst (5) of FIG. 18D, immobilization is made possible by hydrogen bonding between silanols on the silica surface and the para-coordinated sulfonate groups. While a porphyrin ligand is depicted in FIGS. 18A-D, it should be understood that, in other exemplary embodiments, salen ligands may also be attached to a support in this manner.

The ligand can be recovered by washing with polar protic solvent, such as an alcohol, that disrupts the hydrogen bonding network.

In some embodiments, the ligands of formulae (L-A), (L-A1), (L-A2), (L-A3), (L-B), (L-B1), (L-C), (L-C1), (L-C2), (L-C3), (L-D), (L-D1), (L-D2), (L-E), (L-E1), (L-E2), (L-E3), (L-F), (L-F1), (L-F2), (L-F3), (L-F4), (L-G), (L-G1), and (L-G2) may be used in the method described above to produce the corresponding sulfonated ligands and metallated-sulfonated ligands.

For example, when a ligand of formula (L-A) is used in the method, the corresponding sulfonated ligand and the corresponding metallated-sulfonated ligand are as follows:

the ligand:

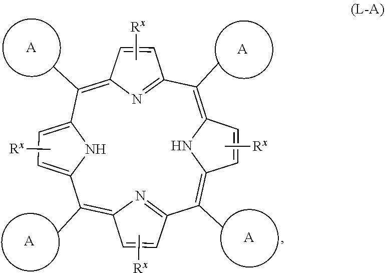

the sulfonated ligand

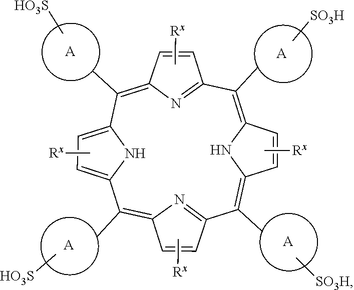

and
the metallated-sulfonated ligand:

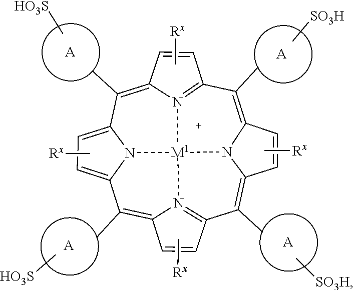

wherein the variables of each formula above are as defined herein.

It should be understood that, although the exemplary sulfonated ligand and corresponding metallated-sulfonated ligand have —SO₃H— moieties at each ring A, in other variations, only one, two or three of the rings A may have the —SO₃H— moiety.

Covalent Tethering

In another aspect, provided is a method of producing a heterogeneous catalyst by: metallating a halo substituted ligand to produce a halo substituted metallated ligand; reacting the halo substituted metallated ligand with an anionic metal carbonyl moiety to produce a metal complex; and combining the metal complex with a solid support comprising aminosiloxane.

With reference to FIGS. 19A-C together, an exemplary reaction scheme is depicted to produce an exemplary heterogeneous catalyst according to such method. As depicted in catalyst (4) of FIG. 19C, the metal complex is attached to the chosen support through reaction of chloro functionality with aminosiloxane that has been grafted onto the solid support. Immobilization is made possible by covalent tethering of the phenyl groups of porphyrin to the amino group attached to the solid support. While a porphyrin ligand is depicted in FIGS. 19A-C, it should be understood that, in other exemplary embodiments, salen ligands may also be attached to a support in this manner.

In other variations, the metal center of the metal complex may also be attached to the solid support.

In some embodiments of the foregoing method, the ligand used is a porphyrin ligand.

In other embodiments, the ligands of formulae (L-A), (L-A1), (L-A2), (L-A3), (L-B), (L-B1), (L-C), (L-C1), (L-C2), (L-C3), (L-D), (L-D1), (L-D2), (L-E), (L-E1), (L-E2), (L-E3), (L-F), (L-F1), (L-F2), (L-F3), (L-F4), (L-G), (L-G1), and (L-G2) may be used in the method described above to produce the corresponding halo substituted ligands and halo substituted metallated ligand.

For example, when a ligand of formula (L-A) is used in the method, the corresponding halo substituted ligand and the corresponding halo substituted metallated ligand are as follows:

the ligand:

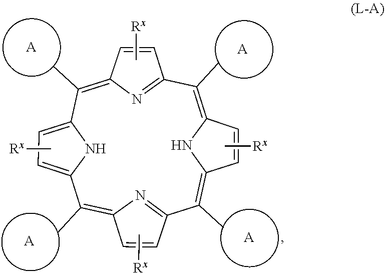

the halo substituted ligand:

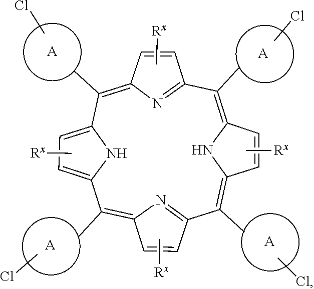

and
the halo substituted metallated ligand:

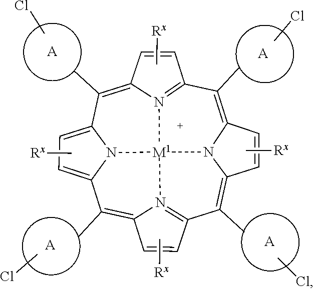

wherein the variables of each formula above are as defined herein.

It should be understood that, although the exemplary halo substituted ligand and corresponding halo substituted metallated ligand have a chloro group at each ring A, in other variations, only one, two or three of the rings A may have the chloro group. Moreover, in other variations, other halo groups, such as fluoro or bromo may be present.

Encapsulation

In yet another aspect, provided is a method of producing a heterogeneous catalyst by: dealuminating a solid support to form a dealuminated solid support, wherein the solid support comprises a plurality of pores; subjecting the dealuminated solid support to ion exchange with a cationic metal; combining a suitable aldehyde compound and a suitable diamine compound to produce a ligand encapsulated within the pores of the solid support; and reacting the encapsulated ligand with an anionic metal carbonyl moiety.

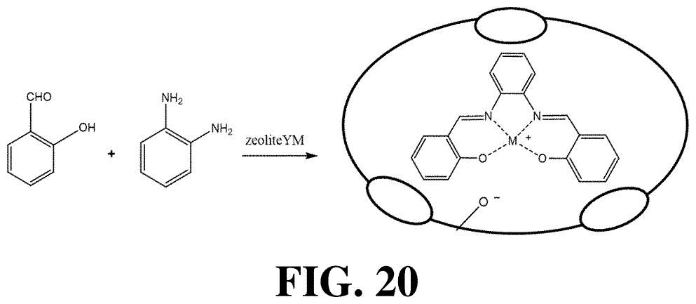

With reference to FIG. 20, an exemplary reaction scheme is depicted to illustrate the building of a salen ligand within the pore of the solid support.

In some embodiments of the foregoing, the ligand used in the method described is a salen ligand.

In other embodiments, the ligands of formulae (L-A), (L-A1), (L-A2), (L-A3), (L-B), (L-B1), (L-C), (L-C1), (L-C2), (L-C3), (L-D), (L-D1), (L-D2), (L-E), (L-E1), (L-E2), (L-E3), (L-F), (L-F1), (L-F2), (L-F3), (L-F4), (L-G), (L-G1), and (L-G2) may be used in the method described above

With respect to the methods and techniques described above, any of the solid supports, ligands, metal atoms, and anionic metal carbonyl moieties may be used as if each and every combination were individually listed.

### Uses of the Heterogeneous Catalysts

The heterogeneous catalysts described herein may be used as catalysts in carbonylation reactions. In certain embodiments, carbonylation of an epoxide of formula

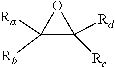

produces a beta-lactone of formula

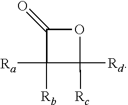

In certain embodiments, each of Rₐ, R_{b}, R_{c}, and R_{d} is independently H, optionally substituted alkyl, optionally substituted alkenyl, optionally substituted cycloalkyl, or optionally substituted aryl. It should be understood that the epoxides and beta-lactones may have asymmetric centers, and may exist in different enantiomeric or diastereomeric forms. All optical isomers and stereoisomers of the compounds of the general formula, and mixtures thereof in any ratio, are considered within the scope of the formula. Thus, any formula provided herein may include (as the case may be) a racemate, one or more enantiomeric forms, one or more diastereomeric forms, one or more atropisomeric forms, and mixtures thereof in any ratio.

“Alkyl” refers to a monoradical unbranched or branched saturated hydrocarbon chain. In some embodiments, alkyl has 1 to 10 carbon atoms (i.e., C₁₋₁₀ alkyl), 1 to 9 carbon atoms (i.e., C₁₋₉ alkyl), 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 5 carbon atoms (i.e., C₁₋₅ alkyl), 1 to 4 carbon atoms (i.e., C₁₋₄ alkyl), 1 to 3 carbon atoms (i.e., C₁₋₃ alkyl), or 1 to 2 carbon atoms (i.e., C₁₋₂ alkyl). Examples of alkyl include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. When an alkyl residue having a specific number of carbon atoms is named, all geometric isomers having that number of carbon atoms may be encompassed; thus, for example, “butyl” can include n-butyl, sec-butyl, isobutyl and t-butyl; “propyl” can include n-propyl and isopropyl.

“Alkenyl” refers to an unsaturated linear or branched monovalent hydrocarbon chain or combination thereof, having at least one site of olefinic unsaturation (i.e., having at least one moiety of the formula C═C). In some embodiments, alkenyl has 2 to 10 carbon atoms (i.e., C₂₋₁₀ alkenyl). The alkenyl group may be in “cis” or “trans” configurations, or alternatively in “E” or “Z” configurations. Examples of alkenyl include ethenyl, allyl, prop-1-enyl, prop-2-enyl, 2-methylprop-1-enyl, but-1-enyl, but-2-enyl, but-3-enyl, isomers thereof, and the like.

“Cycloalkyl” refers to a carbocyclic non-aromatic group that is connected via a ring carbon atom. Examples of cycloalkyl include cyclopropyl, cyclobutyl, cyclopentyl, cyclohexyl, and the like.

“Aryl” refers to a monovalent aromatic carbocyclic group of from 6 to 18 annular carbon atoms having a single ring or a ring system having multiple condensed rings. Examples of aryl include phenyl, naphthyl and the like.

The term “optionally substituted” means that the specified group is unsubstituted or substituted by one or more substituent groups. Examples of substituents may include halo, —OSO₂R₂, —OSiR₄, —OR, C═CR₂, —R, —OC(O)R, —C(O)OR, and —C(O)NR₂, wherein R is independently H, optionally substituted alkyl, optionally substituted alkenyl, or optionally substituted aryl. In some embodiments, R is independently unsubstituted alkyl, unsubstituted alkenyl, or unsubstituted aryl. In some embodiments, R is independently H, methyl (Me), ethyl (Et), propyl (Pr), butyl (Bu), benzyl (Bn), allyl, phenyl (Ph), or a haloalkyl. In certain embodiments, substituents may include F, Cl, —OSO₂Me, —OTBS (where “TBS” is tert-butyl(dimethyl)silyl)), —OMOM (where “MOM” is methoxymethyl acetal), —OMe, —OEt, —OiPr, —OPh, —OCH₂CHCH₂, —OBn, —OCH₂(furyl), —OCF₂CHF₂, —C═CH₂, —OC(O)Me, —OC(O)ₙPr, —OC(O)Ph, —OC(O)C(Me)CH₂, —C(O)OMe, —C(O)OnPr, —C(O)NMe₂, —CN, -Ph, —C₆F₅, —C₆H₄OMe, and —OH.

In one variation, three of Rₐ, R_{b}, R_{c}, and R_{d} are H, and the remaining Rₐ, R_{b}, R_{c}, and R_{d} is optionally substituted alkyl, optionally substituted alkenyl, optionally substituted cycloalkyl, or optionally substituted aryl. In one variation, three of Rₐ, R_{b}, R_{c}, and R_{d} are H, and the remaining Rₐ, R_{b}, R_{c}, and R_{d} is unsubstituted alkyl, or alkyl substituted with a substituent selected from the group consisting of halo, —OSO₂R₂, —OSiR₄, —OR, C═CR₂, —R, —OC(O)R, —C(O)OR, and —C(O)NR₂, wherein R is independently H, Me, Et, Pr, Bu, Bn, allyl, and Ph.

In one variation, two of Rₐ, R_{b}, R_{c}, and R_{d} are H, and the remaining two of Rₐ, R_{b}, R_{c}, and R_{d} are optionally substituted alkyl. In one variation, two of Rₐ, R_{b}, R_{c}, and R_{d} are H, one of the remaining Rₐ, R_{b}, R_{c}, and R_{d} is optionally substituted alkyl, and one of the remaining Rₐ, R_{b}, R_{c}, and R_{d} is optionally substituted aryl. In one variation, two of Rₐ, R_{b}, R_{c}, and R_{d} are H, one of the remaining Rₐ, R_{b}, R_{c}, and R_{d} is optionally substituted alkyl, and one of the remaining Rₐ, R_{b}, R_{c}, and R_{d} is optionally substituted alkenyl. In one variation, two of Rₐ, R_{b}, R_{c}, and R_{d} are H, one of the remaining Rₐ, R_{b}, R_{c}, and R_{d} is optionally substituted alkyl, and one of the remaining Rₐ, R_{b}, R_{c}, and R_{d} is optionally substituted cycloalkyl. In one variation, two of Rₐ, R_{b}, R_{c}, and R_{d} are H, one of the remaining Rₐ, R_{b}, R_{c}, and R_{d} is optionally substituted alkenyl, and one of the remaining Rₐ, R_{b}, R_{c}, and R_{d} is optionally substituted aryl.

In certain embodiments, Rₐ, R_{b}, R_{c}, and R_{d} are H. In certain embodiments, Rₐ, R_{b}, and R_{c} are H, and R_{d} is optionally substituted alkyl. In certain embodiments, R_{d}, R_{b}, and R_{c} are H, and Rₐ is optionally substituted alkyl. In certain embodiments, Rₐ, R_{b}, and R_{c} are H, and R_{d} is optionally substituted alkenyl. In certain embodiments, R_{d}, R_{b}, and R_{c} are H, and Rₐ is optionally substituted alkenyl. In certain embodiments, Rₐ, R_{b}, and R_{c} are H, and R_{d} is optionally substituted cycloalkyl. In certain embodiments, R_{d}, R_{b}, and R_{c} are H, and Rₐ is optionally substituted cycloalkyl. In certain embodiments, Rₐ, R_{b}, and R_{c} are H, and R_{d} is optionally substituted aryl. In certain embodiments, R_{d}, R_{b}, and R_{c} are H, and Rₐ is optionally substituted aryl.

In certain embodiments, Rₐ and R_{b} are optionally substituted alkyl, and R_{c} and R_{d} are H. In certain embodiments, R_{c} and R_{d} are optionally substituted alkyl, and Rₐ and R_{b} are H. In certain embodiments, Rₐ and R_{b} are taken together to form an optionally substituted ring. In certain embodiments, R_{c} and R_{d} are taken together to form an optionally substituted ring. In certain embodiments, the optionally substituted ring is a carbocyclic non-aromatic ring containing from 3 to 10 carbon atoms. In certain embodiments, the carbocyclic non-aromatic ring contains at least one site of olefinic unsaturation.

In certain embodiments, Rₐ and R_{d} are taken together to form an optionally substituted ring. In certain embodiments, the optionally substituted ring is a carbocyclic non-aromatic ring containing from 3 to 10 carbon atoms. In certain embodiments, the carbocyclic non-aromatic ring contains at least one site of olefinic unsaturation.

In certain embodiments, Rₐ and R_{d} are each independently optionally substituted alkyl, and R_{b} and R_{c} are H. In certain embodiments, Rₐ is optionally substituted alkyl, R_{d} is optionally substituted aryl, and R_{b} and R_{c} are H. In certain embodiments, R_{d} is optionally substituted alkyl, Rₐ is optionally substituted aryl, and R_{b} and R_{c} are H. In certain embodiments, Rₐ is optionally substituted alkenyl, R_{d} is optionally substituted aryl, and R_{b} and R_{c} are H. In certain embodiments, R_{d} is optionally substituted alkenyl, Rₐ is optionally substituted aryl, and R_{b} and R_{c} are H. In certain embodiments, Rₐ is optionally substituted alkyl, R_{d} is optionally substituted alkenyl, and R_{b} and R_{c} are H. In certain embodiments, R_{d} is optionally substituted alkyl, Rₐ is optionally substituted alkenyl, and R_{b} and R_{c} are H.

Bio-Content

The combination of epoxide and carbon monoxide in the presence of the heterogeneous catalysts described herein produce at least one beta-lactone and/or beta-lactone derivative. In some variations, the beta-lactones and beta-lactone derivatives may have a bio-content of at least 10%, at least 20%, at least 30%, at least 40%, at least 50%, at least 60%, at least 70%, at least 80%, at least 90%, at least 95%, at least 99%, or 100%.

The terms bio-content and bio-based content mean biogenic carbon also known as bio-mass derived carbon, carbon waste streams, and carbon from municipal solid waste. In some variations, bio-content (also referred to as “bio-based content”) can be determined based on the following:
Bio-content or Bio-based content=[Bio (Organic) Carbon]/[Total (Organic) Carbon]*100%, as determined by ASTM D6866 (Standard Test Methods for Determining the Bio-based (biogenic) Content of Solid, Liquid, and Gaseous Samples Using Radiocarbon Analysis).

The ASTM D6866 method allows for the determination of the bio-based content of materials using radiocarbon analysis by accelerator mass spectrometry, liquid scintillation counting, and isotope mass spectrometry. When nitrogen in the atmosphere is struck by an ultraviolet-light-produced neutron, it loses a proton and forms carbon that has a molecular weight of 14, which is radioactive. This ¹⁴C is immediately oxidized into carbon dioxide, and represents a small, but measurable fraction of atmospheric carbon. Atmospheric carbon dioxide is cycled by green plants to make organic molecules during photosynthesis. The cycle is completed when the green plants or other forms of life metabolize the organic molecules, producing carbon dioxide which is then able to return back to the atmosphere. Virtually all forms of life on Earth depend on this green plant production of organic molecules to produce the chemical energy that facilitates growth and reproduction. Therefore, the ¹⁴C that exists in the atmosphere becomes part of all life forms and their biological products. These renewably based organic molecules that biodegrade to carbon dioxide do not contribute to global warming because no net increase of carbon is emitted to the atmosphere. In contrast, fossil fuel-based carbon does not have the signature radiocarbon ratio of atmospheric carbon dioxide. See WO 2009/155086.

The application of ASTM D6866 to derive a “bio-based content” is built on the same concepts as radiocarbon dating, but without use of the age equations. The analysis is performed by deriving a ratio of the amount of radiocarbon (¹⁴C) in an unknown sample to that of a modern reference standard. The ratio is reported as a percentage, with the units “pMC” (percent modern carbon). If the material being analyzed is a mixture of present day radiocarbon and fossil carbon (containing no radiocarbon), then the pMC value obtained correlates directly to the amount of bio-based material present in the sample. The modern reference standard used in radiocarbon dating is a NIST (National Institute of Standards and Technology) standard with a known radiocarbon content equivalent approximately to the year AD 1950. The year AD 1950 was chosen because it represented a time prior to thermonuclear weapons testing which introduced large amounts of excess radiocarbon into the atmosphere with each explosion (termed “bomb carbon”). The AD 1950 reference represents 100 pMC. “Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. The distribution of bomb carbon has gradually decreased over time, with today's value being near 107.5 pMC. As a result, a fresh biomass material, such as corn, could result in a radiocarbon signature near 107.5 pMC.

Petroleum-based carbon does not have the signature radiocarbon ratio of atmospheric carbon dioxide. Research has noted that fossil fuels and petrochemicals have less than about 1 pMC, and typically less than about 0.1 pMC, for example, less than about 0.03 pMC. However, compounds derived entirely from renewable sources have at least about 95 percent modern carbon (pMC), they may have at least about 99 pMC, including about 100 pMC.

In some embodiments, the products described herein are obtained from renewable sources. In some variations, renewable sources include sources of carbon and/or hydrogen obtained from biological life forms that can replenish itself in less than one hundred years.

In some embodiments, the products described herein have at least one renewable carbon. In some variations, renewable carbon refers to a carbon obtained from biological life forms that can replenish itself in less than one hundred years.

In some embodiments, the products described herein are obtained from recycled sources. In some variations, recycled sources include sources of carbon and/or hydrogen recovered from a previous use in a manufactured article.

In some embodiments, the products described herein have at least one recycled carbon. In some variations, recycled carbon refers to a carbon recovered from a previous use in a manufactured article.

Combining fossil carbon with present day carbon into a material will result in a dilution of the present day pMC content. By presuming that 107.5 pMC represents present day bio-based materials and 0 pMC represents petroleum derivatives, the measured pMC value for that material will reflect the proportions of the two component types. A material derived 100% from present day biomass would give a radiocarbon signature near 107.5 pMC. If that material were diluted with 50% petroleum derivatives, it would give a radiocarbon signature near 54 pMC.

A bio-based content result is derived by assigning 100% equal to 107.5 pMC and 0% equal to 0 pMC. In this regard, a sample measuring 99 pMC will give an equivalent bio-based content result of 93%.

Assessment of the materials described herein according to the present embodiments is performed in accordance with ASTM D6866 revision 12 (i.e. ASTM D6866-12). In some embodiments, the assessments are performed according to the procedures of Method B of ASTM-D6866-12. The mean values encompass an absolute range of 6% (plus and minus 3% on either side of the bio-based content value) to account for variations in end-component radiocarbon signatures. It is presumed that all materials are present day or fossil in origin and that the desired result is the amount of bio-based carbon “present” in the material, not the amount of bio-material “used” in the manufacturing process.

Other techniques for assessing the bio-based content of materials are described in U.S. Pat. Nos. 3,885,155, 4,427,884, 4,973,841, 5,438,194, and 5,661,299, and WO 2009/155086.

In certain embodiments, the heterogeneous catalysts described herein may be used as catalysts in the carbonylation of an epoxide from Column A of Table A below to produce the respective beta-lactone from Column B.

TABLE A

| Column A | Column B |
|---|---|
| 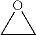 | 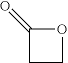 |
| 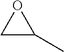 | 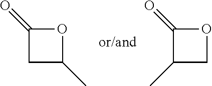 |
| 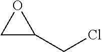 | 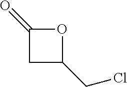 |
| 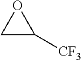 | 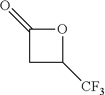 |
| 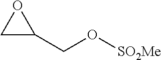 | 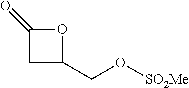 [SMILES: CS(=O)(=O)OCC1CC(=O)O1] |
| 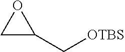 [SMILES: CC(C)(C)[Si](C)(C)OCC1CO1] | 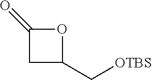 [SMILES: CC(C)(C)[Si](C)(C)OCC1CC(=O)O1] |
| 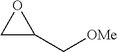 | 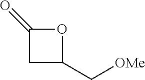 |
| 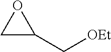 | 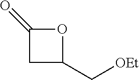 |
| 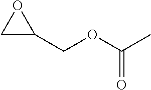 | 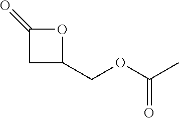 |
| 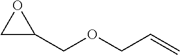 | 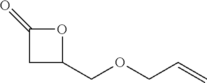 |
| 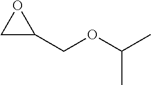 | 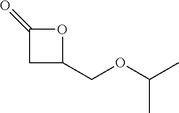 |
| 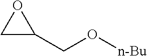 | 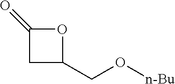 |
| 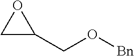 [SMILES: c1ccc(COCC2CO2)cc1] | 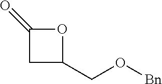 [SMILES: O=C1CC(COCc2ccccc2)O1] |
| 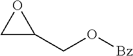 | 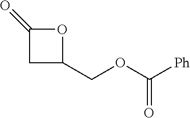 [SMILES: O=C1CC(COC(=O)c2ccccc2)O1] |
| 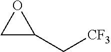 | 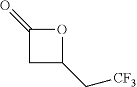 |
| 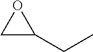 | 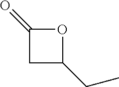 |
| 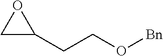 [SMILES: c1ccc(COCCC2CO2)cc1] | 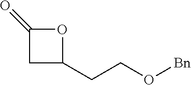 [SMILES: O=C1CC(CCOCc2ccccc2)O1] |
| 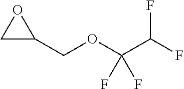 [SMILES: FC(F)C(F)(F)OCC1CO1] | 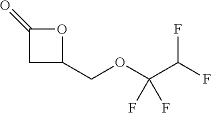 [SMILES: O=C1CC(COC(F)(F)C(F)F)O1] |
| 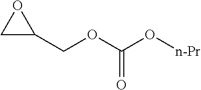 | 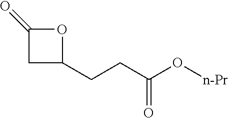 |
| 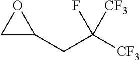 [SMILES: FC(F)(F)C(F)(CC1CO1)C(F)(F)F] | 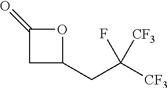 [SMILES: O=C1CC(CC(F)(C(F)(F)F)C(F)(F)F)O1] |
| 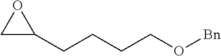 [SMILES: c1ccc(COCCCCC2CO2)cc1] | 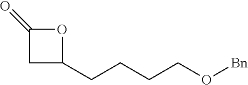 [SMILES: O=C1CC(CCCCOCc2ccccc2)O1] |
| 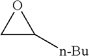 | 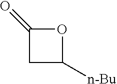 |
| 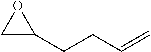 | 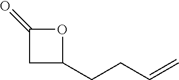 |
| 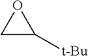 | 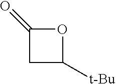 |
| 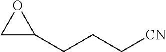 | 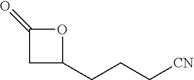 |
| 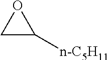 | 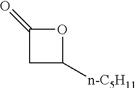 |
| 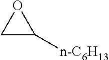 | 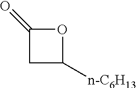 |
| 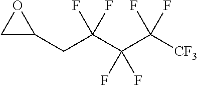 [SMILES: FC(F)(F)C(F)(F)C(F)(F)C(F)(F)CC1CO1] | 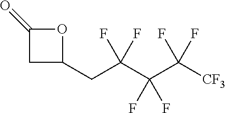 [SMILES: O=C1CC(CC(F)(F)C(F)(F)C(F)(F)C(F)(F)F)O1] |
| 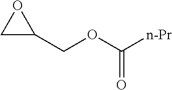 | 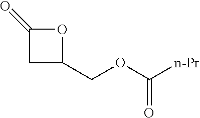 |
| 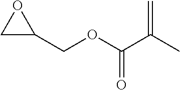 | 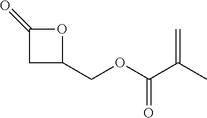 |
| 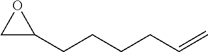 | 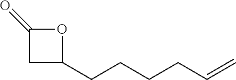 |
| 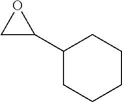 | 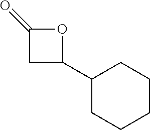 [SMILES: O=C1CC(C2CCCCC2)O1] |
| 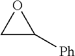 | 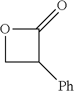 [SMILES: O=C1OCC1c1ccccc1] |
| 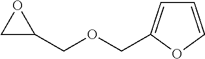 [SMILES: c1coc(COCC2CO2)c1] | 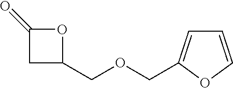 [SMILES: O=C1CC(COCc2ccco2)O1] |
| 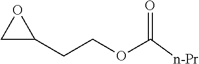 | 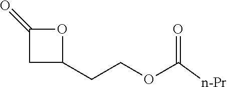 |
| 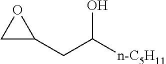 | 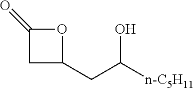 |
| 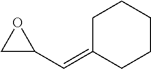 | 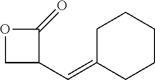 [SMILES: O=C1OCC1C=C1CCCCC1] |
| 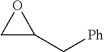 | 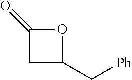 [SMILES: O=C1CC(Cc2ccccc2)O1] |
| 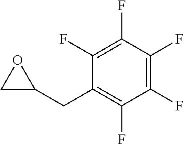 [SMILES: Fc1c(F)c(F)c(CC2CO2)c(F)c1F] | 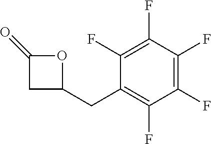 [SMILES: O=C1CC(Cc2c(F)c(F)c(F)c(F)c2F)O1] |
| 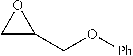 [SMILES: c1ccc(OCC2CO2)cc1] | 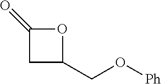 [SMILES: O=C1CC(COc2ccccc2)O1] |
| 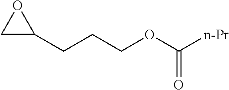 | 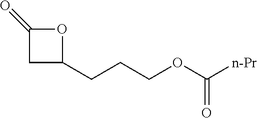 |
| 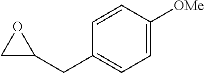 [SMILES: COc1ccc(CC2CO2)cc1] | 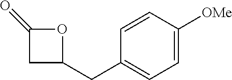 [SMILES: COc1ccc(CC2CC(=O)O2)cc1] |
| 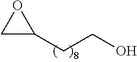 | 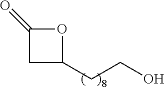 |
| 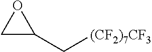 | 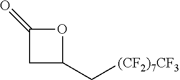 |
| 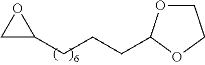 | 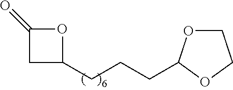 |
| 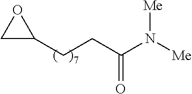 | 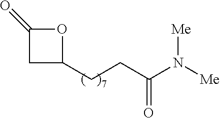 |
| 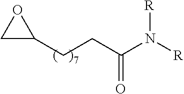 | 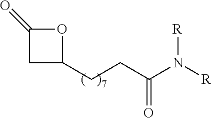 |
| 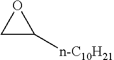 | 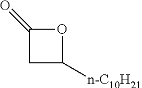 |
| 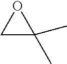 | 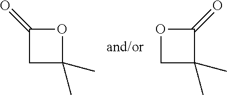 |
| 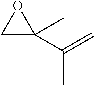 | 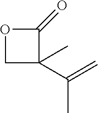 |
| 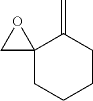 | 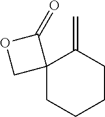 |
| 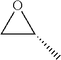 | 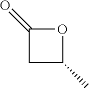 |
| 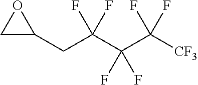 [SMILES: FC(F)(F)C(F)(F)C(F)(F)C(F)(F)CC1CO1] | 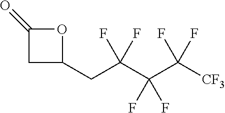 [SMILES: O=C1CC(CC(F)(F)C(F)(F)C(F)(F)C(F)(F)F)O1] |
| 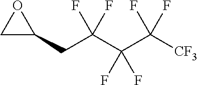 [SMILES: FC(F)(F)C(F)(F)C(F)(F)C(F)(F)C[C@H]1CO1] | 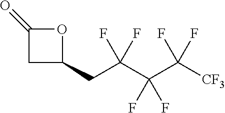 [SMILES: O=C1C[C@H](CC(F)(F)C(F)(F)C(F)(F)C(F)(F)F)O1] |
| 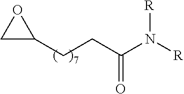 | 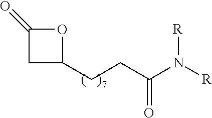 |
| 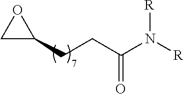 | 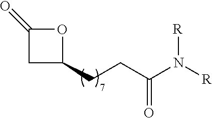 |
| 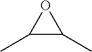 | 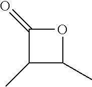 |
| 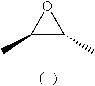 | 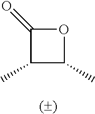 |
| 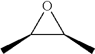 | 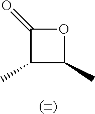 |
| 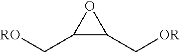 | 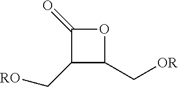 |
| 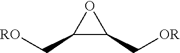 | 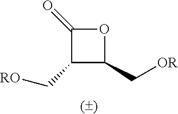 |
| 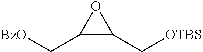 | 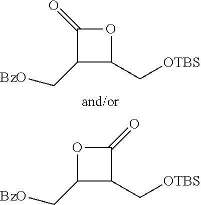 |
| 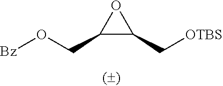 | 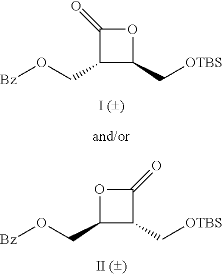 |
| 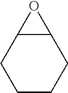 | 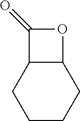 |
| 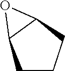 | 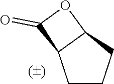 |
| 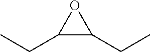 | 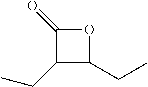 |
| 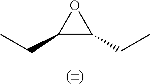 | 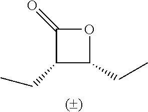 |
| 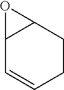 | 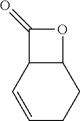 |
| 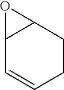 | 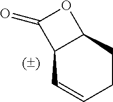 |
| 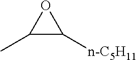 | 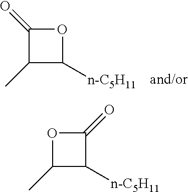 |
| 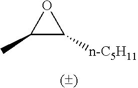 | 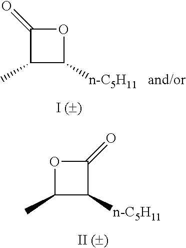 |
| 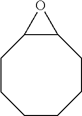 | 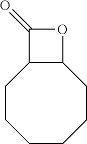 [SMILES: O=C1OC2CCCCCCC12] |
| 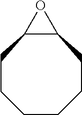 | 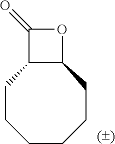 [SMILES: O=C1O[C@H]2CCCCCC[C@H]12] |
| 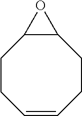 | 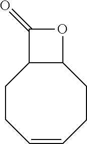 [SMILES: O=C1OC2CC/C=C\CCC12] |
| 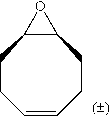 | 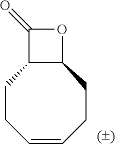 [SMILES: O=C1O[C@H]2CC/C=C\CC[C@H]12] |
| 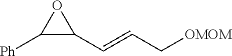 | 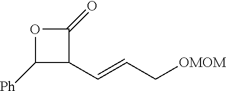 |
| 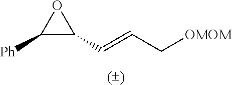 | 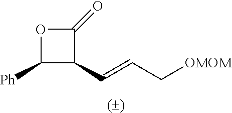 |
| 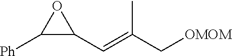 | 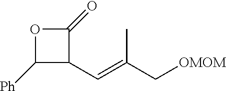 |
| 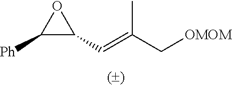 | 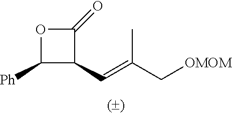 |
| 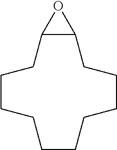 [SMILES: C1CCCCCC2OC2CCCC1] | 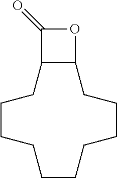 [SMILES: O=C1OC2CCCCCCCCCCC12] |
| 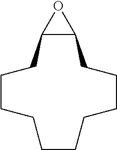 [SMILES: C1CCCCC[C@H]2O[C@H]2CCCC1] | 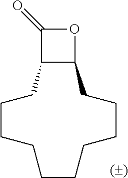 [SMILES: O=C1O[C@H]2CCCCCCCCCC[C@H]12] |
| 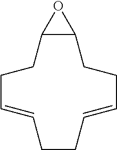 [SMILES: C1=C/CCC2OC2CC/C=C/CC/1] | 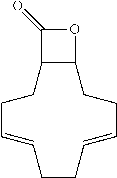 [SMILES: O=C1OC2CC/C=C/CC/C=C/CCC12] |
| 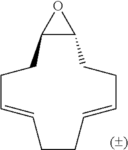 [SMILES: C1=C/CC[C@H]2O[C@@H]2CC/C=C/CC/1] | 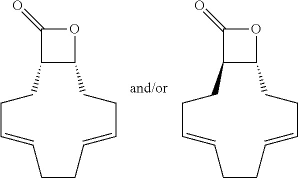 [SMILES: O=C1O[C@@H]2CC/C=C/CC/C=C/CC[C@H]12 ; O=C1O[C@@H]2CC/C=C/CC/C=C/CC[C@@H]12] |
| 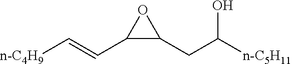 | 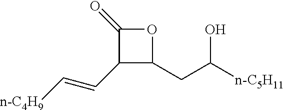 |
| 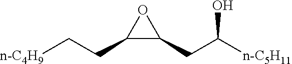 | 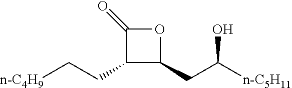 |
| 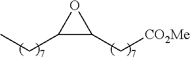 | 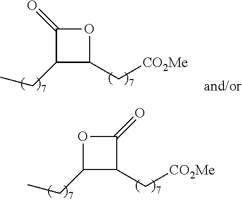 |
| 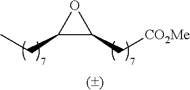 | 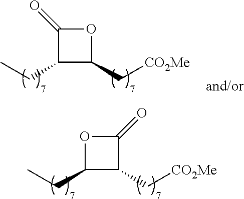 |
| 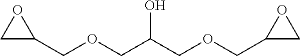 [SMILES: OC(COCC1CO1)COCC1CO1] | 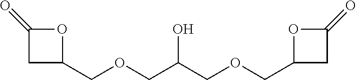 [SMILES: O=C1CC(COCC(O)COCC2CC(=O)O2)O1] |
| 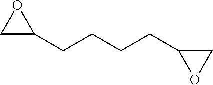 | 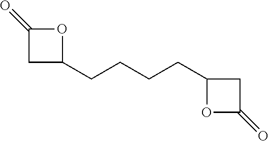 [SMILES: O=C1CC(CCCCC2CC(=O)O2)O1] |
| 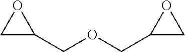 | 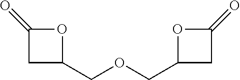 [SMILES: O=C1CC(COCC2CC(=O)O2)O1] |

In some aspects, provided is a method comprising reacting an epoxide with carbon monoxide in the presence of a heterogeneous catalyst, as described herein, to produce a beta-lactone product. In some embodiments, provided is a method comprising carbonylating an epoxide in the presence of a heterogeneous catalyst, as described herein, to produce a beta-lactone product. In some variations, the heterogeneous catalysts used are single-crystalline materials with a large degree of ordering to help prevent leeching of Co(CO)₄⁻ or Co₂(CO)₆⁻ (as the case may be) from the structure.

In other aspects, provided is a method, comprising: reacting an epoxide with carbon monoxide in the presence of a heterogeneous catalyst, as described herein, and a solvent to produce a product stream, wherein the product stream comprises a beta-lactone product and the solvent; and purifying the product stream by distillation to separate the product stream into a solvent recycle stream and a purified beta-lactone stream, wherein the solvent recycle stream comprises the solvent, and wherein the purified beta-lactone stream comprises the beta-lactone product. In some variations, provided is a method, comprising: carbonylating an epoxide in the presence of a heterogeneous catalyst, as described herein, and a solvent to produce a product stream, wherein the product stream comprises a beta-lactone product and the solvent; and purifying the product stream by distillation to separate the product stream into a solvent recycle stream and a purified beta-lactone stream, wherein the solvent recycle stream comprises the solvent, and wherein the purified beta-lactone stream comprises the beta-lactone product.

In other aspects, provided is a system comprising:
a beta-lactone production system, comprising:
   a carbon monoxide source;
   an epoxide source;
   optionally a solvent source;
   a carbonylation reactor, wherein the carbonylation reactor is a fixed or fluid bed reactor comprising:
      a heterogeneous catalyst as described herein,
      at least one inlet to receive carbon monoxide from the carbon monoxide source, epoxide from the epoxide source, and solvent from the solvent source (if present),
      an outlet to output a beta-lactone stream, wherein the beta-lactone stream comprises a beta-lactone product and solvent (if present).

In some variations, a solvent source is not present in the system. In other variations, the solvent source is present in the system.

In yet other aspects, provided is a system comprising:
a beta-lactone production system, comprising:
   a carbon monoxide source;
   an epoxide source;
   a solvent source;
   a carbonylation reactor, wherein the carbonylation reactor is a fixed or fluid bed reactor comprising:
      a heterogeneous catalyst as described herein,
      at least one inlet to receive carbon monoxide from the carbon monoxide source, epoxide from the epoxide source, and solvent from the solvent source, and
      an outlet to output a beta-lactone stream, wherein the beta-lactone stream comprises a beta-lactone product and solvent; and a beta-lactone purification system, comprising:
at least one distillation column configured to receive the beta-lactone stream from the carbonylation reactor, and separate the beta-lactone stream into a solvent recycle stream and a purified beta-lactone stream,
   wherein the solvent recycle stream comprises solvent, and
   wherein the purified beta-lactone stream comprises the beta-lactone product.

In one variation of the methods and systems described herein, the epoxide is ethylene oxide, and the beta-lactone product is beta-propiolactone. The beta-propiolactone may be used as a precursor to produce polypropiolactone and/or acrylic acid.

In some variations of the foregoing, provided herein are systems and methods using the heterogeneous catalysts described herein for the production of acrylic acid from ethylene oxide and carbon monoxide on an industrial scale. In certain variations, the methods and systems described herein are suitable for the production of acrylic acid on a scale of 25 kilo tons per annum (“KTA”). In some variations, the systems are configured to produce acrylic acid using the heterogeneous catalysts described herein in a continuous process, and further feedback loops to continually produce acrylic acid.

Further, in some variations, the systems provided herein further include various purification systems to produce acrylic acid of high purity. For example, the systems provided herein may be configured to remove carbonylation solvent and by-products (e.g., acetaldehyde, succinic anhydride, and acrylic acid dimer level) to achieve acrylic acid with a purity of at least 99.5%, at least 99.6%, at least 99.7%, at least 99.8%, or at least 99.9%.

In other variations, the systems provided herein are also configured to recycle various starting materials and acrylic acid precursors, such as beta-propiolactone. For example, the systems may include one or more recycle systems to isolate unreacted ethylene oxide, unreacted carbon monoxide, and carbonylation solvent.

In yet other variations, the systems provided herein are also configured to manage and integrate heat produced. The carbonylation reaction to produce beta-propiolactone and the polymerization reaction to produce polypropiolactone are exothermic. Thus, the heat generated from the exothermic unit operations, such as the carbonylation reactor and polymerization reactor can be captured and used for cooling in endothermic unit operations, such as the distillation apparatus and thermolysis reactor. For example, in some variations of the methods and systems provided herein, steam may be generated in heat transfer equipment (e.g., shell and tube heat exchanger and reactor cooling jacket) via a temperature gradient between process fluid and water/steam. This steam can be used for heat integration between exothermic and endothermic unit operations. In other variations of the systems and methods provided herein, other suitable heat transfer fluids may be used.

In other variations, heat integration may be achieved by combining certain unit operations. For example, heat integration may be achieved by combining polymerization of beta-propiolactone and vaporization of the solvent (e.g., THF) from the distillation column within a single unit operation. In such a configuration, the heat liberated from the beta-propiolactone polymerization reaction is used directly to vaporize the solvent in the distillation apparatus, and the output of the unit produces polypropiolactone. In other variations, the heat liberated from the polymerization reaction can be exported to other systems at the same production site.

With reference to FIG. 2, an exemplary system to produce acrylic acid from carbon monoxide and ethylene oxide is depicted. Carbon monoxide (CO), ethylene oxide (EO) and carbonylation solvent are fed into a beta-propiolactone production system, as depicted in FIG. 2. In some variations, the reactor in the system for producing beta-propiolactone is a fluid or fixed bed reactor. In other variations, the reactor contains a heterogeneous catalyst as described herein. Such beta-propiolactone production system is typically configured to produce a liquid product stream of beta-propiolactone. This beta-propiolactone product stream is fed to an EO/CO separator, depicted as the flash tank in FIG. 2, where unreacted ethylene oxide and unreacted carbon monoxide may be separated and recycled for use in the reactor. The beta-propiolactone product stream is then fed from the EO/CO separator to a distillation column in FIG. 2, which is configured to separate ethylene oxide, carbon monoxide, and by-products from the solvent recycle stream, which is depicted as a tetrahydrofuran (THF) recycle stream. The system in FIG. 2 depicts the use of THF as the carbonylation solvent, but it should be understood that in other variations, other suitable solvents may be used. The purified beta-propiolactone stream and polymerization catalyst are fed into a polypropiolactone production system, depicted as a plug flow reactor in FIG. 2. The polypropiolactone production system is configured to produce a polypropiolactone product stream, which can be fed into a thermolysis reactor to produce acrylic acid.

It should be understood, however, that while FIG. 2 depicts an exemplary acrylic acid production system, variations of this production system are envisioned. It should also be understood that FIG. 2 depicts an exemplary system for producing beta-propiolactone from ethylene oxide, the system may be configured to use other epoxides and produce corresponding beta-lactones as provided in Table A above.

Additionally, in other exemplary embodiments of the systems described herein, various unit operations depicted in FIG. 2 may be combined or omitted. In some variations, polymerization (e.g., to form polypropiolactone from beta-propiolactone) and depolymerization (e.g., to form acrylic acid from depolymerization of polypropiolactone) may be combined (e.g. by catalytic or reactive distillation) may be combined, or the EO/CO separator may be omitted.

Further, it should be understood that in other exemplary embodiments of systems described herein, additional unit operations may be employed. For example, in some embodiments, one or more heat exchangers may be incorporated into the systems to manage and integrate heat produced in the system.

Provided herein are various systems configured for the commercial production of polypropiolactone and acrylic acid. In some configurations, polypropiolactone and acrylic acid are produced at the same geographical location. In other configurations, polypropiolactone is produced in one location and shipped to a second location where acrylic acid is produced.

In other variations, beta-propiolactone may be polymerized to produce polypropiolactone by way of complete conversion of beta-propiolactone. In such a variation, there may not be a need for additional apparatus in the system to isolate and recycle beta-propiolactone to the polymerization reactor. In other variations, the conversion of beta-propiolactone is not complete. Unreacted beta-propiolactone may be separated from the polypropiolactone product stream and the recovered beta-propiolactone may be recycled back to the polymerization reactor.

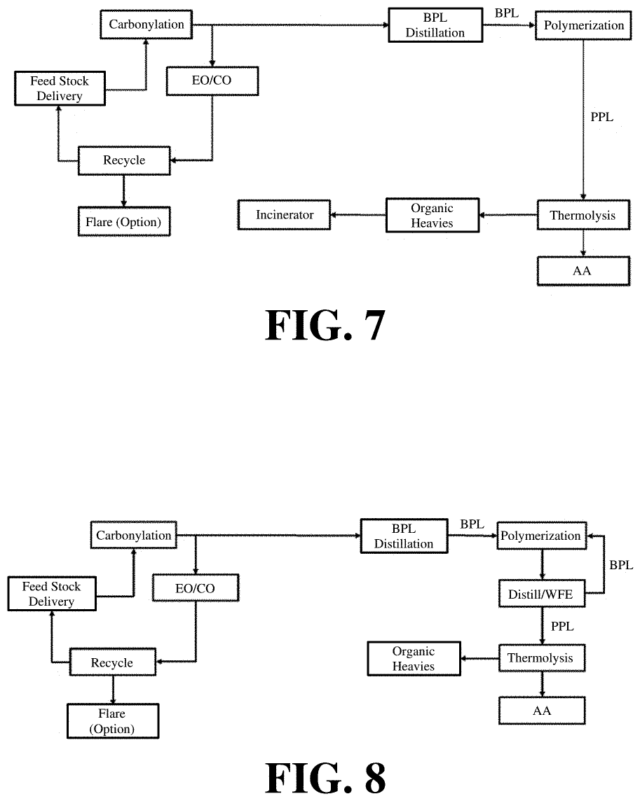

For example, FIG. 7 depicts an exemplary system wherein the PPL product stream and the AA product stream are produced at the same location, and the polypropiolactone production system is configured to achieve complete conversion of BPL to PPL. The BPL production system (labeled ‘Carbonylation’ in FIG. 7) typically includes a carbon monoxide (CO) source, an ethylene oxide (EO) source, a solvent source, and a carbonylation reactor which contains the carbonylation catalyst. In certain variations, the carbonylation reactor is configured to receive carbon monoxide (CO), ethylene oxide (EO), and solvent from a CO source, an EO source, and a solvent source (collectively labeled ‘Feed Stock Delivery’ in FIG. 7). The carbon monoxide, ethylene oxide, carbonylation solvent, and carbonylation catalyst may be obtained by any commercially available sources, or any commercially available methods and techniques known in the art.

In some variations, the CO, EO, and solvent are essentially water and oxygen free. In one variation, the solvent from the solvent source, the EO from the EO source, and the CO from the CO source have a concentration of water and oxygen less than about 500 ppm, less than about 250 ppm, less than about 100, or less than about 50 ppm.

Any suitable carbonylation solvents may be used. In some embodiments, the carbonylation solvent comprises tetrahydrofuran, hexane, or a combination thereof. In other embodiments, the carbonylation solvent comprises an ether, a hydrocarbon, or a combination thereof. In yet other embodiments, the carbonylation solvent comprises tetrahydrofuran, tetrahydropyran, 2,5-dimethyl tetrahydrofuran, sulfolane, N-methyl pyrrolidone, 1,3 dimethyl-2-imidazolidinone, diglyme, triglyme, tetraglyme, diethylene glycol dibutyl ether, isosorbide ethers, methyl tert-butyl ether, diethylether, diphenyl ether, 1,4-dioxane, ethylene carbonate, propylene carbonate, butylene carbonate, dibasic esters, diethyl ether, acetonitrile, ethyl acetate, propyl acetate, butyl acetate, 2-butanone, cyclohexanone, toluene, difluorobenzene, dimethoxy ethane, acetone, or methylethyl ketone, or any combination thereof. In one variation, the carbonylation solvent comprises tetrahydrofuran.

The carbonylation reactor may be configured to receive EO from the EO source at any rate, temperature, or pressure described herein. Additionally, the carbonylation reactor may be configured to receive CO from the CO source at any rate, temperature, or pressure described herein. The carbonylation reactor may be also configured to receive solvent at any rate, temperature, or pressure described herein.

In some embodiments, the pressure in the carbonylation reactor is about 900 psig, and the temperature is about 70° C. In certain variations, the reactor is equipped with an external cooler (heat exchanger). In some variations, the carbonylation reaction achieves a selectivity of BPL above 99%.

With reference again to the exemplary system in FIG. 7, a beta-propiolactone product stream exits the outlet of the carbonylation reactor. The beta-propiolactone product stream comprises BPL, solvent, unreacted EO and CO, and by-products, such as acetaldehyde by-product (ACH) and succinic anhydride (SAH). The beta-propiolactone product stream may have any concentration of BPL, solvent, EO, ACH, and SAH described herein.

With reference again to the exemplary system in FIG. 7, the beta-propiolactone product stream is output from an outlet of the carbonylation reactor and enters an inlet of the ethylene oxide and carbon monoxide separator (labeled ‘EO/CO’ in FIG. 7). In one embodiment, the ethylene oxide and carbon monoxide separator is a flash tank. The majority of the ethylene oxide and carbon monoxide is recovered from the carbonylation reaction stream and can be recycled back to the carbonylation reactor as a recycled ethylene oxide stream and a recycled carbon monoxide stream (labeled ‘Recycle’ in FIG. 7), or sent for disposal (labeled ‘Flare’ in FIG. 7). In some embodiments, at least 10% of the ethylene oxide and 80% of the carbon monoxide in the carbonylation reaction stream is recovered. The recycled carbon monoxide stream can also include unreacted ethylene oxide, secondary reaction product acetaldehyde, BPL, and the remainder solvent.

In some variations, the ethylene oxide and carbon monoxide are disposed of using a method other than flare. For example, in one embodiment, the ethylene oxide and carbon monoxide recovered from the beta-propiolactone product stream are disposed of using incineration.

With reference again to the exemplary system in FIG. 7, the beta-propiolactone product stream may enter the inlet of the BPL purification system (labeled ‘BPL Distillation’ in FIG. 7). In one variation, the BPL purification system comprises one or more distillation columns operating at or below atmospheric pressure configured to produce a recovered solvent stream, and a production stream comprising purified BPL. The pressure is selected in such a way to achieve the temperature that reduces the decomposition of BPL. In some embodiments, the one or more distillation columns are operated at a pressure of about 0.15 bara and a temperature between about 90° C. and about 120° C. In some embodiments, the distillation system is configured to produce a recycled solvent stream essentially free of ethylene oxide, carbon monoxide, acetaldehyde, and succinic anhydride.

With reference again to the exemplary system in FIG. 7, the recovered solvent stream exits an outlet of the BPL purification system and may be fed back to the carbonylation reactor. In some variations, the concentration of H₂O and O₂ is reduced in the recycled solvent stream prior to being fed to the carbonylation reactor. The recovered solvent stream may have any concentration of H₂O and O₂ described herein when fed back to the carbonylation reactor. For example, in some embodiments, the concentration of H₂O and O₂ is less than about 500 ppm, less than about 250 ppm, less than about 100 ppm, or less than about 50 ppm when fed back into the carbonylation reactor.

With reference again to the exemplary system in FIG. 7, the production stream comprising purified BPL exits the outlet of the BPL purification system. The production stream is essentially free of solvent, ethylene oxide, carbon monoxide, acetaldehyde, and succinic anhydride. In some embodiments, the remainder of the production stream includes secondary reaction products such as succinic anhydride, and leftover solvent (e.g., THF).

The production stream enters an inlet of the polypropiolactone production system. In the exemplary system depicted in FIG. 7, the polypropiolactone production system comprises a polymerization reactor (labeled ‘Polymerization’ in FIG. 7). The polypropiolactone production system is configured to receive and output streams at any rate, concentration, temperature, or pressure described herein. For example, in one embodiment, the inlet to the polymerization process can include about 2000 kg/hr BPL to about 35000 kg/hr BPL.

With reference again to the exemplary system in FIG. 7, the polypropiolactone production system is configured to operate in a continuous mode and achieves complete conversion of BPL in the production stream to PPL. A PPL product stream (labeled ‘PPL’ in FIG. 7) exits an outlet of the polypropiolactone production system, and comprises PPL.

With reference again to the exemplary system in FIG. 7, the PPL product stream enters an inlet of the thermolysis reactor. The PPL product stream may have any concentration of compounds, temperature, or pressure described herein. A thermolysis reactor is configured to convert the PPL stream to an AA product stream. In some embodiments, the temperature of the thermolysis reactor is between 200° C. and 300° C. and the pressure is between 0.2 bara and 5 bara.

Traces of high boiling organic impurities (labeled ‘Organic Heavies’ in FIG. 7) are separated from the AA stream, exit an outlet of the thermolysis reactor, and are sent to the incinerator for disposal (labeled ‘Incinerator’ in FIG. 7).

An AA product stream exits an outlet of the thermolysis reactor for storage or further processing. The AA product stream comprises essentially pure AA. The AA product stream may exit an outlet of the thermolysis reactor at any rate, concentration, temperature, or pressure described herein. The remainder of the AA product stream can include secondary reaction products such as succinic anhydride or acetaldehyde and left over solvent such as THF. In some embodiments, the AA product stream can have a temperature between about 20° C. to about 60° C. In some embodiments, the AA product stream can be at a pressure of about 0.5 to about 1.5 bara.

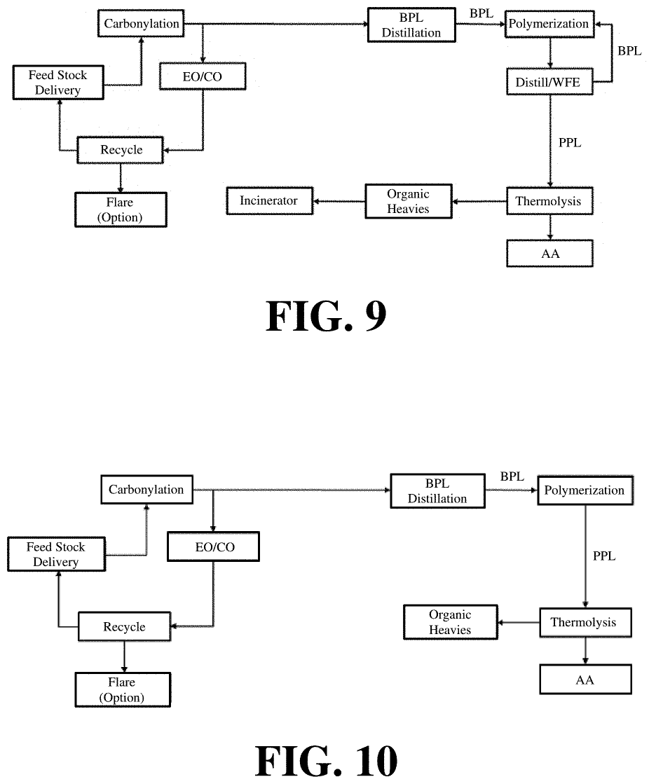

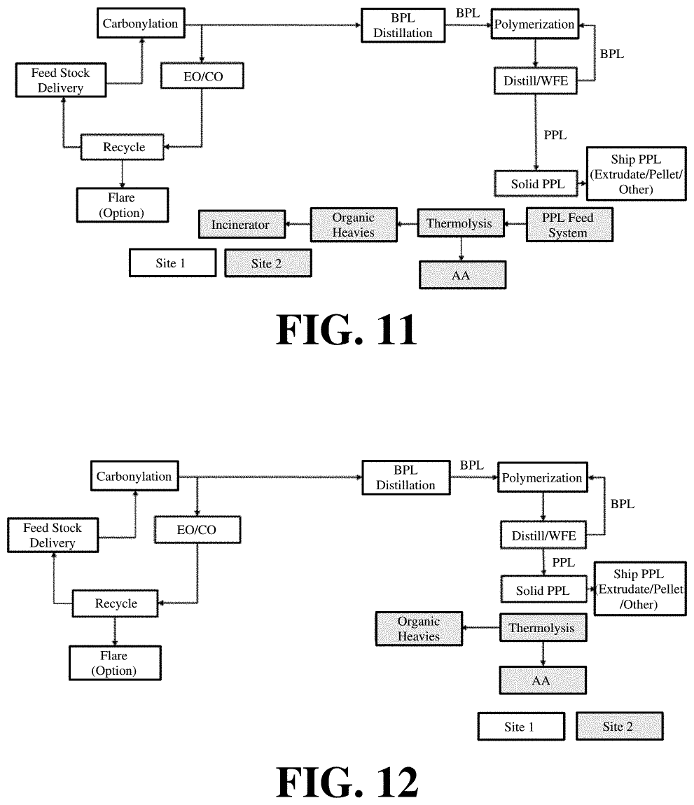

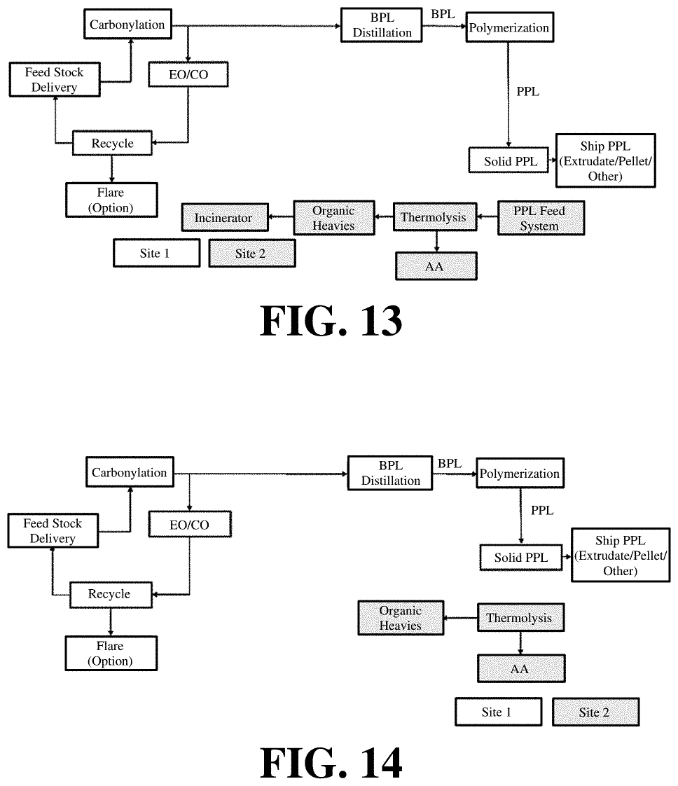

Other variations in the configurations of the systems are provided in FIGS. 8-14. Each of the unit operations in the production systems for acrylic acid and precursors thereof are also described in further detail below.

Beta-Lactone Production System (i.e., Carbonylation Reaction System)

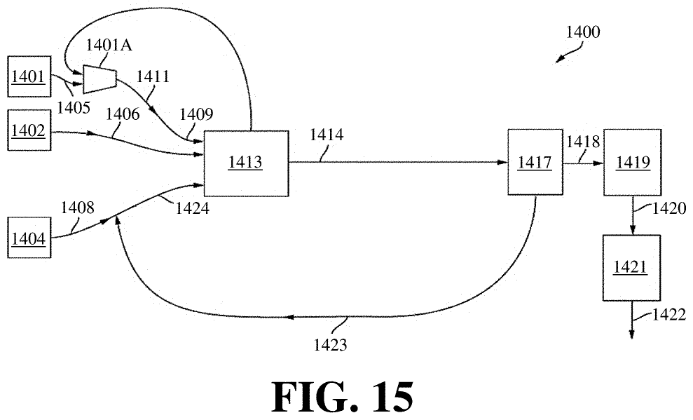

FIG. 15 illustrates an exemplary embodiment of the production system disclosed herein. FIG. 15 contains carbonylation reaction system 1413 (i.e., beta-propiolactone production system), BPL purification system 1417, polymerization reaction system 1419, and thermolysis system 1421.

In the carbonylation reaction system, ethylene oxide (an exemplary epoxide) can be converted to beta-propiolactone (an exemplary beta-lactone) by a carbonylation reaction, as depicted in the reaction scheme below.

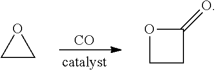

Water and oxygen can damage the carbonylation catalyst. The feed streams (i.e., EO, CO, and optionally solvent) to the carbonylation reaction reactor, which contains the carbonylation catalyst, should be substantially dry (i.e., have a water content below 50 ppm) and be oxygen free (i.e., have an oxygen content below 20 ppm). As such, the feed streams and/or storage tanks and/or feed tank can have sensors on them in order to determine the composition of the stream/tank to make sure that they have a low enough oxygen and water content. In some embodiments, the feed streams can be purified such as by adsorption to reduce the water and oxygen content in the streams fed to the carbonylation reaction system. In some embodiments, prior to running the production system, the tubes, apparatuses, and other flow paths can be purged with an inert gas or carbon monoxide to minimize exposure to oxygen or water in the production system.

FIG. 15 includes ethylene oxide source 1402 that can feed fresh ethylene oxide in ethylene oxide stream 1406 to carbonylation reaction system inlet 1409. Inlet 1409 can be one inlet to the carbonylation reaction system or multiple inlets. Ethylene oxide can be fed as a liquid using a pump or any other means known to those of ordinary skill in the art. In addition, the ethylene oxide source can be maintained under an inert atmosphere.

FIG. 15 also includes solvent source 1404 that can feed solvent to the carbonylation reaction system. The solvent may be selected from any solvents described herein, and mixtures of such solvents. In some variations, the solvent is an organic solvent. In certain variations, the solvent is an aprotic solvent. In some embodiments, the solvent includes dimethylformamide, N-methyl pyrrolidone, tetrahydrofuran, toluene, xylene, diethyl ether, methyl-tert-butyl ether, acetone, methylethyl ketone, methyl-iso-butyl ketone, butyl acetate, ethyl acetate, dichloromethane, and hexane, and mixtures of any two or more of these. In general, polar aprotic solvents or hydrocarbons are suitable for this step.

Additionally, in one variation, beta-lactone may be utilized as a co-solvent. In other variations, the solvent may include ethers, hydrocarbons and non protic polar solvents. In some embodiments, the solvent includes tetrahydrofuran (“THF”), sulfolane, N-methyl pyrrolidone, 1,3 dimethyl-2-imidazolidinone, diglyme, triglyme, tetraglyme, diethylene glycol dibutyl ether, isosorbide ethers, methyl tert-butyl ether, diethylether, diphenyl ether, 1,4-dioxane, ethylene carbonate, propylene carbonate, butylene carbonate, dibasic esters, diethyl ether, acetonitrile, ethyl acetate, dimethoxy ethane, acetone, and methylethyl ketone. In other embodiments, the solvent includes tetrahydrofuran, tetrahydropyran, 2,5-dimethyl tetrahydrofuran, sulfolane, N-methyl pyrrolidone, 1,3 dimethyl-2-imidazolidinone, diglyme, triglyme, tetraglyme, diethylene glycol dibutyl ether, isosorbide ethers, methyl tert-butyl ether, diethylether, diphenyl ether, 1,4-dioxane, ethylene carbonate, propylene carbonate, butylene carbonate, dibasic esters, diethyl ether, acetonitrile, ethyl acetate, propyl acetate, butyl acetate, 2-butanone, cyclohexanone, toluene, difluorobenzene, dimethoxy ethane, acetone, and methylethyl ketone. In certain variations, the solvent is a polar donating solvent. In one variation, the solvent is THF.

Referring again to the exemplary system depicted in FIG. 15, in some embodiments, solvent feed 1424 can supply solvent to the carbonylation reaction system inlet 1409. Solvent can be fed to the carbonylation reaction system using a pump. In addition, the solvent streams, sources, storage tanks, etc., can be maintained under an inert or CO atmosphere. In some embodiments, the solvent feed that supplies solvent to the carbonylation reaction system can include solvent 1408 from fresh solvent source 1404, and recycled solvent 1423 from the BPL purification system. In some embodiments, the recycled solvent from the BPL purification system can be stored in a make-up solvent reservoir. In some embodiments, the solvent feed that supplies solvent to the carbonylation reaction system can include solvent from the make-up solvent reservoir. In some embodiments, solvent can be purged from the system. In some embodiments, the purged solvent can be solvent from the recycled solvent of the BPL purification system. In some embodiments, solvent from the fresh solvent source is also stored into the make-up solvent reservoir to dilute the recycled solvent from the BPL purification system with fresh solvent. In some embodiments, fresh solvent is fed from the fresh solvent source to the make-up solvent reservoir prior to entering the carbonylation reaction system. In some embodiments, solvent from the fresh solvent source and the BPL purification system can be purified by operations such as adsorption to remove oxygen and water that can inhibit the carbonylation catalyst. In some embodiments, the amount of oxygen and/or water in all streams entering the carbonylation reaction system is less than about 500 ppm, less than about 250 ppm, less than about 100, less than about 50 ppm, or less than about 20 ppm.

In certain variations, the carbonylation reaction systems and methods for carbonylation described herein do not use a solvent.

The beta-propiolactone production system may further include other feed sources. For example, in one variation, the beta-propiolactone production system further includes a Lewis base additive source.

In some embodiments, a Lewis base additive may be added to the carbonylation reactor. In certain embodiments, such Lewis base additives can stabilize or reduce deactivation of the catalysts. In some embodiments, the Lewis base additive is selected from the group consisting of phosphines, amines, guanidines, amidines, and nitrogen-containing heterocycles. In some embodiments, the Lewis base additive is a hindered amine base. In some embodiments, the Lewis base additive is a 2,6-lutidine; imidazole, 1-methylimidazole, 4-dimethylaminopyridine, trihexylamine and triphenylphosphine.

The exemplary system depicted in FIG. 15 also includes carbonylation product stream 1414, BPL purified stream 1418, PPL product stream 1420, and AA product stream 1422.

In some embodiments, the carbonylation reaction system can include at least one reactor for the carbonylation reaction. In some embodiments, the carbonylation system can include multiple reactors in series and/or parallel for the carbonylation reaction. In some variations, the reactor is a fixed or fluid bed reactor with a heterogeneous catalyst comprising any of the heterogeneous catalysts described herein.

All inlets and outlets to the carbonylation reaction system can include sensors that can determine the flowrate, composition (especially water and/or oxygen content), temperature, pressure, and other variables known to those of ordinary skill in the art. In addition, the sensors can be connected to control units that can control the various streams (i.e., feed controls) in order to adjust the process based on the needs of the process determined by the sensor units. Such control units can adjust the quality as well as the process controls of the system.

In some variations, the reactor in the beta-propiolactone production system is configured to further receive one or more additional components. In certain embodiments, the additional components comprise diluents which do not directly participate in the chemical reactions of ethylene oxide. In certain embodiments, such diluents may include one or more inert gases (e.g., nitrogen, argon, helium and the like) or volatile organic molecules such as hydrocarbons, ethers, and the like. In certain embodiments, the reaction stream may comprise hydrogen, carbon monoxide of carbon dioxide, methane, and other compounds commonly found in industrial carbon monoxide streams. In certain embodiments, such additional components may have a direct or indirect chemical function in one or more of the processes involved in the conversion of ethylene oxide to beta-propiolactone and various end products. Additional reactants can also include mixtures of carbon monoxide and another gas. For example, as noted above, in certain embodiments, carbon monoxide is provided in a mixture with hydrogen (e.g., Syngas).

Because the carbonylation reaction is exothermic, the reactors used can include an external circulation loop for reaction mass cooling. In some embodiments, the reactors can also include internal heat exchangers for cooling. For example, in the case of a shell and tube type reactor, the reactors can flow through the tube part of the reactor and a cooling medium can flow through the shell of the reactor or vice versa. Heat exchanger systems can vary depending on layout, reactor selection, as well as physical location of the reactor. The reactors can employ heat exchangers outside of the reactors in order to do the cooling/heating or the reactors can have an integrated heat exchanger such as a tube and shell reactor. For example, the reactor can utilize a layout for heat rejection by pumping a portion of the reaction fluid through an external heat exchanger. In some embodiments, heat can be removed from the reactor by using a coolant in a reactor jacket, one or more internal cooling coils, lower temperature feeds and/or recycle streams, an external heat exchange with pump around loop, and/or other methods known by those of ordinary skill in the art. In addition, the reactors may have multiple cooling zones with varying heat transfer areas and/or heat transfer fluid temperatures and flows.

In some embodiments, the heat produced in the reaction system can be reduced by adding additional solvent to the reaction system in order to dilute the reactants, decreasing the reactants in the reaction system, and/or decreasing the amount of catalyst in the reaction system.

The type of reactor employed and the type of heat exchanger employed (either external or integrated) can be a function of various chemistry considerations (e.g., reaction conversions, by-products, etc.), degree of exotherm produced, and the mixing requirements for the reaction.

Since carbonylation reactions are exothermic reactions and the BPL purification system and thermolysis requires energy, it is possible to integrate at least some of the components between the carbonylation reaction system and the BPL purification system and/or thermolysis system. For example, steam can be formed in a heat exchanger of the carbonylation reaction system and transported to the BPL purification system for heating a distillation column for example. In addition, the BPL purification system and the carbonylation reaction system may be integrated into a single system or unit so that the heat produced from the carbonylation reaction can be used in the BPL purification system (in an evaporator or distillation column). The steam can be generated in a heat exchanger via a temperature gradient between reaction fluids and water/steam of the heat exchanger. Steam can be used for heat integration between exothermic units (carbonylation reaction, polymerization reaction) and endothermic units (BPL purification system's columns/evaporators and thermolysis reaction). In some embodiments, steam is only used for heat management and integration and will not be introduced directly into the production processes.

As previously described, water and oxygen can affect the carbonylation catalyst. As such, oxygen and water intrusion into the carbonylation system should also be minimized. As such, the reactor can have a mag drive, a double mechanical seal, and/or materials of construction that are compatible with the reactants and products of the carbonylation reaction but not permeable to atmosphere. In some embodiments, the materials of construction of the reactor include metals. In some embodiments, the metals can be stainless steel. In some embodiments, the metals can be carbon steel. In some embodiments, the metals can be metal alloys such as nickel alloys. In some embodiments, the metals are chosen when compatibility or process conditions dictate, e.g., high chloride content or if carbon steel catalyzes EO decomposition. In some embodiments, everything up until the polymerization reaction system can include carbon steel. One of the benefits of carbon steel over stainless steel is its cost. In some embodiments, the metals can have a surface finish so as to minimize polymer nucleation sites. The materials of construction of the reactor can also include elastomer seals. In some embodiments, the elastomer seals are compatible with the reactants and products of the carbonylation reaction but not permeable to the atmosphere. Examples of elastomer seals include but are not limited to Kalrez 6375, Chemraz 505, PTFE encapsulated Viton, and PEEK. The materials of construction of external parts of the carbonylation reaction system can be compatible with the environment, for example, compatible with sand, salty water, not heat absorbing, and can protect the equipment from the environment.

In some embodiments, the carbonylation reaction system is operated so as to minimize or mitigate PPL formation prior to the polymerization reaction system. In some embodiments, the carbonylation reaction system is operated so as to avoid catalyst decomposition.

In some embodiments, the carbonylation reactor(s) can have a downstream flash tank with a reflux condenser to separate unreacted carbon monoxide as a recycled carbon monoxide stream from the carbonylation reaction system. As previously described, the recycled carbon monoxide stream can be sent to a CO compressor and/or combined with a fresh carbon monoxide feed prior to being sent back into the carbonylation reaction system. The flash tank can separate most of the CO to avoid its separation downstream. In some embodiments, excess gas is removed or purged from the reactor itself and thus a flash tank is not necessary.

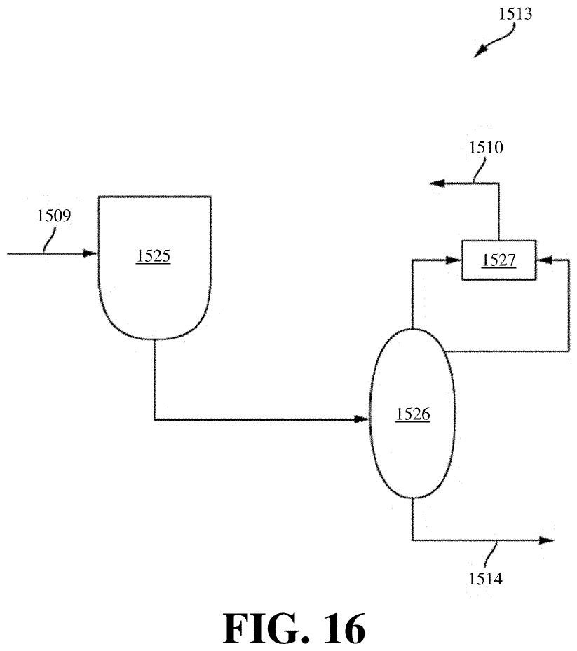

FIG. 16 illustrates an exemplary embodiment of a carbonylation reaction system disclosed herein. Carbonylation reaction system 1513 can include carbonylation reaction system inlet 1509 for carbonylation reactor 1525. As previously described, the inlet can be made up of multiple inlets or feeds into the reaction system. In addition, carbonylation reaction system 1513 includes flash tank 1526 with condenser 1527. Flash tank 1526 and condenser 1527 separate the reactor product stream into recycled carbon monoxide stream 1510 and beta-propiolactone product stream 1514.

BPL Purification System (and Solvent Recycle)

The beta-propiolactone product stream can be fed to the BPL purification system. The BPL purification system can separate BPL into a BPL purified stream from low-boiling impurities before it enters the polymerization reaction system, where high purity BPL can be required. In some embodiments, the BPL purified stream can have at least about 90 wt % BPL, at least about 95 wt % BPL, at least about 98 wt % BPL, at least about 99 wt % BPL, at least about 99.3 wt % BPL, at least about 99.5 wt % BPL, at least about 99.8 wt %, or at least about 99.9 wt %. In some embodiments, the BPL purified stream can have at most about 1 wt % solvent, at most about 0.5 wt % solvent, or at most about 0.1 wt % solvent. In some embodiments, the BPL purification system can also create a solvent recycle stream. In some embodiments, the BPL purification system can separate the BPL from the other components in the stream such as solvent, unreacted ethylene oxide, unreacted carbon monoxide, secondary reaction product acetaldehyde, and secondary reaction product succinic anhydride In some embodiments, the temperature in the BPL purification system can be at most about 150° C., at most about 125° C., at most about 115° C., at most about 105° C., or at most about 100° C. When BPL is exposed to temperatures greater than 100° C., the BPL can potentially decompose or be partially polymerized. Accordingly, the BPL can be purified without being exposed to temperatures of about 150° C., 125° C., 115° C., 105° C., or 100° C.

In some embodiments, the separation is performed by exploiting the boiling point differential between the beta-propiolactone and the other components of the carbonylation product stream, primarily the solvent. In some embodiments, the boiling point of the solvent is lower than the boiling point of the beta-propiolactone. In some embodiments, the solvent is volatilized (e.g., evaporated) from the BPL purification feed along with other lighter components (e.g., ethylene oxide & acetaldehyde), leaving behind BPL, other heavier compounds (e.g., catalyst and succinic anhydride) and some leftover solvent from the BPL purification feed. In some embodiments, this includes exposing the BPL purification feed to reduced pressure. In some embodiments, this includes exposing BPL purification feed to increased temperature. In some embodiments, this includes exposing the BPL purification feed to both reduced pressure and increased temperature.

In some embodiments, the separation may be effected in a sequence of steps, each operating at an independent temperature and pressure. For example, in one embodiment, two steps may be used to obtain a more effective separation of beta-propiolactone, or a separate separation step may be used to isolate certain reaction by-products. In some embodiments, when a mixture of solvents is used, multiple separation steps may be required to remove particular solvents, individually or as a group, and effectively isolate the beta-propiolactone.

In certain embodiments, the separation of the beta-propiolactone from the BPL purification feed is performed in two stages. In some embodiments the process includes a preliminary separation step to remove one or more components of the BPL purification feed having boiling points below that of the beta-propiolactone product.

In some embodiments, the preliminary separation step includes separating the BPL purification feed into a gas stream comprising ethylene oxide, solvent, and BPL (and potentially carbon monoxide, acetaldehyde, and/or BPL); and a liquid stream comprising beta-propiolactone (and potentially succinic anhydride and/or solvent). In the second step of separation, the liquid stream is further separated into a beta-propiolactone stream comprising beta-propiolactone, a solvent stream comprising solvent, and potentially succinic anhydride purge stream. The gas stream can also be further separated into a solvent stream comprising solvent, a light gases stream comprising solvent and ethylene oxide (and potentially acetaldehyde), and a liquid BPL stream comprising BPL and solvent. The liquid BPL stream can join with the liquid stream prior to separation of the liquid stream and form a combined feed to the second separation step. In some embodiments, the solvent stream from the second separation step and/or the solvent stream from the gas stream separation can form the solvent recycle stream which can be fed to the carbonylation reaction system or to a solvent reservoir.

In some embodiments where one or more solvents with a boiling point lower than that of the beta-propiolactone are present, the lower boiling solvent may be volatilized (e.g., evaporated) from the BPL purification feed in a preliminary separation step, leaving behind a mixture comprising catalyst, beta-propiolactone, other solvents (if any) and other compounds in the BPL purification stream which is then further treated to separate the beta-propiolactone stream.

In certain embodiments where the separation is performed in two stages, the first step of separation comprises exposing the reaction stream to mildly reduced pressure to produce the gas stream and the liquid stream. In certain embodiments where the separation is performed in two stages, the gas stream can be returned to the carbonylation step.

In certain embodiments, the separation of the beta-propiolactone from the BPL purification feed is performed in three stages. In the first step of separation, the BPL purification feed is separated into a gaseous stream comprising ethylene oxide, solvent, and BPL (and potentially carbon monoxide and/or acetaldehyde); and a liquid stream comprising solvent and beta-propiolactone (and potentially succinic anhydride). In the second step of separation, the gaseous stream is separated into a solvent side stream comprising solvent; a light gas stream comprising ethylene oxide and solvent (and potentially carbon monoxide and/or acetaldehyde); and second liquid stream comprising solvent and BPL. In the third step of separation, the second liquid stream and the first liquid stream are combined and separated into a gaseous solvent stream comprising solvent, a purified BPL stream comprising BPL, and potentially a succinic anhydride purge stream. In some embodiments, the solvent side stream and/or the gaseous solvent stream can be used as the solvent recycle stream for use in the carbonylation reaction system or can be stored in a solvent storage tank.

In certain embodiments where the separation is performed in three stages, the first step of separation comprises exposing the BPL purification feed to atmospheric pressure. In certain embodiments where the separation is performed in three stages, the second step of separation comprises exposing the gaseous stream to atmospheric pressure. In certain embodiments where the separation is performed in three stages, the third step of separation comprises exposing the gaseous stream to a vacuum or reduced pressure. In certain embodiments, the reduced pressure is between about 0.05-0.25 bara. In certain embodiments, the reduced pressure is between about 0.1-0.2 bara or about 0.15 bara.

In certain embodiments, the separation of the beta-propiolactone from the BPL purification feed is performed in four stages. In the first step of separation, the BPL purification feed is separated into a gaseous stream comprising ethylene oxide, solvent, and BPL (and potentially carbon monoxide and/or acetaldehyde); and a liquid stream comprising solvent, beta-propiolactone (and potentially succinic anhydride). In the second step of separation, the gaseous stream is separated into a solvent side stream comprising solvent; a light gas stream comprising ethylene oxide and solvent (and potentially carbon monoxide and/or acetaldehyde); and second liquid stream comprising solvent and BPL. In the third step of separation, the second liquid stream and the first liquid stream are combined and separated into a gaseous solvent stream comprising solvent, a purified BPL stream comprising BPL, and potentially a catalyst and succinic anhydride purge stream. In the fourth step of separation, the light gas stream is separated into a third solvent stream comprising solvent and a second light gas stream comprising ethylene oxide (and potentially carbon monoxide and/or acetaldehyde). In some embodiments, the solvent side stream, the gaseous solvent stream, and/or the third solvent stream can be used as the solvent recycle stream for use in the carbonylation reaction system or can be stored in a solvent storage tank.

In certain embodiments where the separation is performed in four stages, the first step of separation comprises exposing the BPL purification feed to atmospheric pressure. In certain embodiments where the separation is performed in four stages, the second step of separation comprises exposing the gaseous stream to atmospheric pressure. In certain embodiments where the separation is performed in four stages, the third step of separation comprises exposing the combined liquid stream to a vacuum or reduced pressure. In certain embodiments, the reduced pressure is between about 0.05-0.25 bara. In certain embodiments, the reduced pressure is between about 0.1-0.2 bara or about 0.15 bara. In certain embodiments where the separation is performed in four stages, the fourth step of separation comprises exposing the light gas stream to atmospheric pressure.

In some embodiments, the BPL purification system can include at least one distillation column to separate BPL from the other components in the post-isolation carbonylation stream. In some embodiments, the BPL purification system includes at least two distillation columns. In some embodiments, the BPL purification system includes at least three distillation columns. In some embodiments, at least one of the distillation columns is a stripping column (i.e., stripper). In some embodiments, at least one of the distillation columns is a vacuum column. In some embodiments, the BPL purification system can include an initial evaporator, wherein the post-isolation carbonylation stream is first fed to an evaporator in the BPL purification system. The evaporator can perform a simple separation between the solvent and the BPL in the post-isolation carbonylation stream. The evaporator can reduce loads on subsequent distillation columns making them smaller. In some embodiments, the evaporator can reduce loads on subsequent distillation columns making them smaller by evaporating solvent in the post-isolation carbonylation stream at about atmospheric pressure and about 100° C.

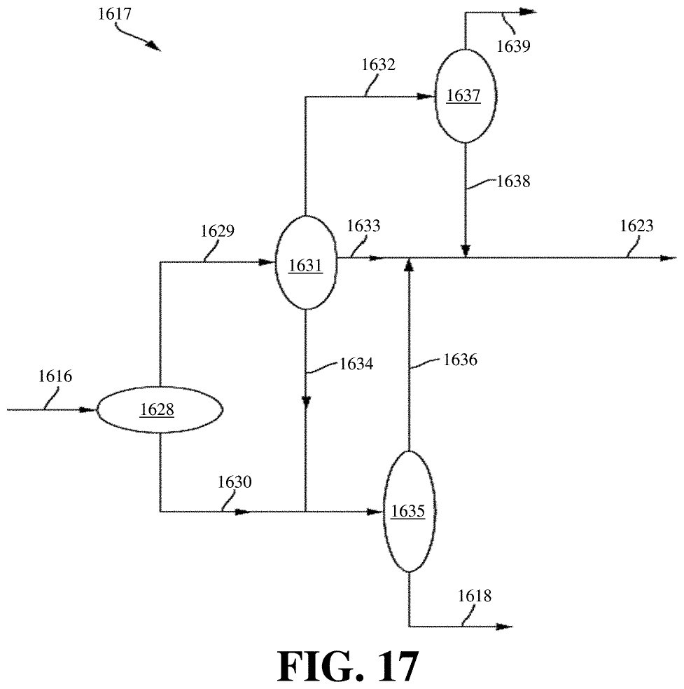

FIG. 17 illustrates an exemplary embodiment of the BPL purification system disclosed herein. In some embodiments, the feed to the BPL purification system can be fed to evaporator 1628. In some embodiments, the evaporator can operate at most about 5 bara, at most about 4 bara, at most about 3 bara, at most about 2 bara, at most about atmospheric pressure (i.e., 1 bara), or at about atmospheric pressure. In some embodiments, the evaporator can operate at a temperature between about 80-120° C., between about 90-100° C., between about 95-105° C., at about 100° C., at most about 100° C., at most about 105° C., at most about 110° C., or at most about 120° C. In some embodiments, the evaporator is a flash tank. Referring again to FIG. 17, in the exemplary system evaporator 1628 can separate the feed into overhead stream 1629 and bottoms stream 1630. Overhead stream 1629 can comprise mainly of THF with low boiling point components (e.g., CO, EO, acetaldehyde) and a small amount of BPL.

Referring again to FIG. 17, in the exemplary system depicted overhead stream 1629 can be sent to solvent purification column 1631. The solvent purification column can be a distillation column. In some embodiments, the solvent purification column can be a stripping column or stripper. In some embodiments, the solvent purification column can operate at most about 5 bara, at most about 4 bara, at most about 3 bara, at most about 2 bara, at most about atmospheric pressure (i.e., 1 bara), or at about atmospheric pressure. In some embodiments, the evaporator can operate at a temperature of at most about 100° C., at most about 105° C., at most about 110° C., or at most about 120° C. In some embodiments, an overhead temperature is maintained at about 20-60° C., about 30-50° C., about 40-50° C., about 44° C. In some embodiments, the solvent purification column can prevent BPL from getting into any vent streams. In some embodiments, solvent purification column can have at least 12 stages with a condenser as stage 1. In some embodiments, solvent purification column can have an internal cooler which can create a side stream. In some embodiments, solvent purification column can have an internal cooler above the side stream withdrawal. In some embodiments, internal cooler can be between stages in the middle of the column. In some embodiments, internal cooler can be between stages 5 and 6 of the solvent purification column. In some embodiments, solvent purification column can separate overhead stream 1629 into overhead stream 1632, bottoms stream 1634, and side stream 1633. Overhead stream 1632 can comprise low boiling components (e.g., EO, CO, acetaldehyde) and around half solvent. Bottoms stream 1634 can comprise mainly BPL and solvent. In some embodiments, solvent purification column can recover at least 90 wt %, at least 95 wt %, at least 98 wt %, at least 99 wt %, or at least 99.5 wt % of BPL from overhead stream 1629 in bottoms stream 1634.

Bottoms stream 1630 and bottoms stream 1634 can be combined and sent to BPL purification column 1635. BPL purification column can be a distillation column. In some embodiments, BPL purification column can be a vacuum column or a column operating under reduced pressure. In some embodiments, the operating pressure of the BPL purification column can be less than atmospheric pressure (1 bara), less than about 0.5 bara, less than about 0.25 bara less than 0.2 bara, less than 0.15 bara, or about 0.15 bara. In some embodiments, the BPL purification column can include a reboiler that can be maintained at most about 120° C., at most about 110° C., at most about 100° C., or about 100° C. In some embodiments, an overhead temperature is maintained at about 5-30° C., about 10-20° C., about 12-16° C., about 14° C.

In some embodiments, BPL purification column can separate the combined bottoms streams 1630 and 1634 into overhead stream 1636 and bottoms stream 1618 (i.e., BPL purified stream 1618). Bottoms stream 1618 can be substantially pure BPL with minimal solvent. In some embodiments, bottoms stream 1618 can also include some heavy components such as succinic anhydride. Succinic anhydride can have some volatility and if accumulated in the sump can produce an undesirable rise in boiling temperature in the reboiler. In some embodiments, succinic anhydride can accumulate in the sump and can be purged from the sump by periodically purging the sump when the succinic anhydride wt % reaches a predefined value (e.g., at least 1 wt %, 2 wt %, 3 wt %, 4 wt %, or 5 wt %). In some embodiments, overhead stream 1636 can have a mass flow rate of about at least about 500 kg/hr, at least about 600 kg/hr, at least about 700 kg/hr, at least about 750 kg/hr at least about 800 kg/hr, or at least about 850 kg/hr. In some embodiments, overhead stream 1636 can have a solvent wt % of at least about 95, at least about 98, at least about 99, at least about 99.1, or at least about 99.5. In some embodiments, overhead stream 1636 can have an ethylene oxide wt % of about 0-3, about 0.2-2, about 0.2-1.5, about 0.5-1, about 0.8, at most about 3, at most about 2, at most about 1, at most about 0.8, at most about 0.5. In some embodiments, overhead stream 1638 can have an acetaldehyde wt % of about 0-0.2, about 0.05-0.15, about 0.1, at most about 0.1, or at most about 0.2.

Overhead stream 1632 can be sent to light gas column 1637 to be separated into overhead stream 1639 and bottoms stream 1638. The light gas column can be a distillation column. In some embodiments, the light gas column can operate at most about 5 bara, at most about 4 bara, at most about 3 bara, at most about 2 bara, at most about atmospheric pressure (i.e., 1 bara), or at about atmospheric pressure. In some embodiments, light gas column can include a partial condenser. In some embodiments, the partial condenser operates at a temperature of at about 0-20° C., about 5-15° C., about 10-15° C., about 10-13° C. In some embodiments, the temperature maintained at the bottom of light gas column is about 20-70° C., about 40-60° C., about 45-55° C., or about 50° C. In some embodiments, the overhead temperature maintained in light gas column can be about −10-10° C., about −5-5° C., about −2-3° C., or about 1° C. Overhead stream 1639 can comprise mostly of the acetaldehyde produced in the carbonylation reaction system as well as low boiling point ethylene oxide. In some embodiments, overhead stream 1639 can be disposed of (e.g., incinerator, flare, etc.) so acetaldehyde does not accumulate in the overall production system.

In some embodiments, side stream 1633, bottoms stream 1638, overhead stream 1636 or combinations thereof can form solvent recycle stream 1623. In some embodiments, side stream 1633, bottoms stream 1638, and overhead stream 1636 can be combined to form solvent recycle stream 1623. In some embodiment, side stream 1633, bottoms stream 1638, and/or overhead stream 1636 can be sent to a solvent recycle tank or storage. In some embodiments, the solvent recycle stream is fed back to the carbonylation reaction system. In some embodiments, the solvent recycle stream fed to the carbonylation reaction system is from the solvent recycle tank or storage. In some embodiments, the solvent streams entering and/or exiting the solvent recycle tank or storage can be purified for example by passing the stream through an absorber to remove potential oxygen and/or moisture from the stream. In some embodiments, the solvent recycle tank or storage can be equipped with sensors to determine the water and/or oxygen content in the storage tank.

Polypropiolactone Production System

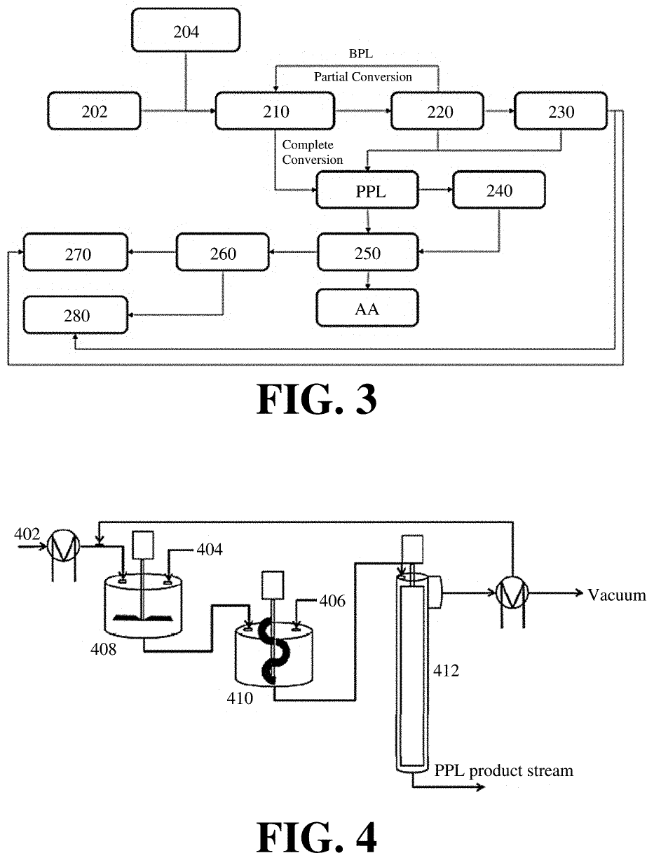

With reference to FIG. 3, the relationship of the polypropiolactone production system with other unit operations, such as the beta-propiolactone purification system and the acrylic acid production system, is depicted.

Beta-propiolactone purification system 202 is configured to feed a beta-propiolactone product stream into polypropiolactone production system 210. Homogeneous catalyst delivery system 204 is configured to feed a homogeneous polymerization catalyst into the polymerization reactor of polypropiolactone production system 210. Polypropiolactone production system 210 is configured to polymerize beta-propiolactone to produce polypropiolactone. Depending on the type of polymerization reactors selected and the configuration of such reactors, as well as the operating conditions (e.g., operating temperature, operating pressure, and residence time) and choice of polymerization catalysts used, the extent of conversion of the beta-propiolactone may be controlled. In some variations, operating temperature is the average temperature of the contents of the reactor.

In some variations, partial conversion of beta-propiolactone to polypropiolactone is achieved, and distillation unit 220 is configured to recycle at least a portion of unreacted beta-propiolactone to polypropiolactone production system 210. In other variations, complete conversion of beta-propiolactone to polypropiolactone is achieved. The polypropiolactone product stream produced from polypropiolactone production system 210 is fed to acrylic acid production system 250, which is configured to produce acrylic acid from the polypropiolactone.

In some variations, unit 240 is configured to receive the polypropiolactone product stream (e.g., in liquid form) from polypropiolactone production system 210, and is configured to pelletize, extrude, flake, or granulate the polypropiolactone product stream.

It should be understood, however, that FIG. 3 provides one exemplary configuration of these unit operations. In other variations, one or more of the unit operations depicted in FIG. 3 may be added, combined or omitted, and the order of the unit operations may be varied as well.

With reference again to FIG. 2, the polypropiolactone production system is configured to produce polypropiolactone by polymerizing beta-propiolactone in the presence of a polymerization catalyst. While FIG. 2 depicts the use of a single plug flow reactor for the polymerization of beta-propiolactone to produce polypropiolactone, other reactor types and reactor configurations may be employed.

In some embodiments, the polypropiolactone production system includes a beta-propiolactone, a polymerization catalyst source, and at least one polymerization reactor.

In certain embodiments, conversion of BPL to PPL is performed in a continuous flow format. In certain embodiments, conversion of BPL to PPL is performed in a continuous flow format in the gas phase. In certain embodiments, conversion of BPL to PPL is performed in a continuous flow format in the liquid phase. In certain embodiments, conversion of BPL to PPL is performed in a liquid phase in a batch or semi-batch format. Conversion of BPL to PPL may be performed under a variety of conditions. In certain embodiments, the reaction may be performed in the presence of one or more catalysts that facilitate the transformation of the BPL to PPL.

In some embodiments, the production stream entering the polymerization process is a gas or a liquid. The conversion of BPL to PPL in the polymerization process may be performed in either the gas phase or the liquid phase and may be performed neat, or in the presence of a carrier gas, solvent, or other diluent.

In certain variations, the operating temperature of the polymerization reactor is maintained at or below the pyrolysis temperature of polypropiolactone.

Any suitable polymerization catalysts may be used to convert the BPL product stream entering the PPL production system into a PPL product stream. In some embodiments, the polymerization catalyst is homogenous with the polymerization reaction mixture. Any suitable homogeneous polymerization catalyst capable of converting the production stream to the PPL product stream may be used in the methods described herein.

The polymerization process may further comprise a polymerization initiator including but not limited to alcohols, amines, polyols, polyamines, and diols, amongst others. Further, a variety of polymerization catalysts may be used in the polymerization process, including by not limited to metals (e.g., lithium, sodium, potassium, magnesium, calcium, zinc, aluminum, titanium, cobalt, etc.) metal oxides, carbonates of alkali- and alkaline earth metals, borates, silicates, of various metals.

In certain embodiments, suitable polymerization catalysts include carboxylate salts of metal ions or organic cations. In some embodiments, a carboxylate salt is other than a carbonate.

In certain embodiments, a polymerization catalyst is combined with the production stream containing BPL. In certain embodiments, the molar ratio of the polymerization catalyst to the BPL in the production stream is about 1:100 polymerization catalyst:BPL to about 25:100 polymerization catalyst:BPL. In certain embodiments, the molar ratio of polymerization catalyst:BPL is about 1:100, 5:100, 10:100, 15:100, 20:100, 25:100, or a range including any two of these ratios.

In certain embodiments, where the polymerization catalyst comprises a carboxylate salt, the carboxylate has a structure such that upon initiating polymerization of BPL, the polymer chains produced have an acrylate chain end. In certain embodiments, the carboxylate ion on a polymerization catalyst is the anionic form of a chain transfer agent used in the polymerization process.

In certain embodiments, the polymerization catalyst comprises a carboxylate salt of an organic cation. In certain embodiments, the polymerization catalyst comprises a carboxylate salt of a cation wherein the positive charge is located at least partially on a nitrogen, sulfur, or phosphorus atom. In certain embodiments, the polymerization catalyst comprises a carboxylate salt of a nitrogen cation. In certain embodiments, the polymerization catalyst comprises a carboxylate salt of a cation selected from the group consisting of: ammonium, amidinium, guanidinium, a cationic form of a nitrogen heterocycle, and any combination of two or more of these. In certain embodiments, the polymerization catalyst comprises a carboxylate salt of a phosphorus cation. In certain embodiments, the polymerization catalyst comprises a carboxylate salt of a cation selected from the group consisting of: phosphonium and phosphazenium. In certain embodiments, the polymerization catalyst comprises a carboxylate salt of a sulfur-containing cation. In certain embodiments, the polymerization catalyst comprises a sulfonium salt.

In some embodiments, the homogeneous polymerization catalyst is a quaternary ammonium salt (for example, tetrabutylammonium (TBA) acrylate, TBA acetate, trimethylphenylammonium acrylate, or trimethylphenylammonium acetate) or a phosphine (for example, tetraphenyl phosphonium acrylate).

In some embodiments, the catalyst is tetrabutylammonium acrylate, iron chloride, TB A acrylate, TBA acetate, trimethylphenylammonium acrylate, trimethylphenylammonium acetate, or tetraphenyl phosphonium acrylate.

With reference to FIG. 4, the polymerization catalyst in the first reactor (408) and the additional polymerization catalyst in the second reactor (410) may be the same or different. For example, in some embodiments, wherein the same catalyst is used in both reactors, concentration of catalyst is not the same in each reactor.

In some embodiments, the homogeneous polymerization catalyst is added to a polymerization reactor as a liquid. In other embodiments it is added as a solid, which then becomes homogeneous in the polymerization reaction. In some embodiments where the polymerization catalyst is added as a liquid, the polymerization catalyst may be added to the polymerization reactor as a melt or in any suitable solvent. For example, in some variations AA, molten PPL or BPL is used as a solvent.

In some embodiments, the solvent for the polymerization catalyst is selected such that the catalyst is soluble, the solvent does not contaminate the product polymer, and the solvent is dry. In some variations, the polymerization catalyst solvent is AA, molten PPL, or BPL. In certain variations, solid PPL is added to a polymerization reactor, heated above room temperature until liquid, and used as the polymerization catalyst solvent. In other embodiments, BPL is added to the polymerization reactor, cooled below room temperature until liquid, and used as the polymerization catalyst solvent.

In some variations, the liquid polymerization catalyst (as a melt or as a solution in a suitable solvent) is prepared in one location, then shipped to a second location where it is used in the polymerization reactor. In other embodiments, the liquid polymerization catalyst (as a melt or as a solution in a suitable solvent) is prepared at the location of the polymerization reactor (for example, to reduce exposure to moisture and/or oxygen).

A liquid polymerization catalyst (as a melt or as a solution in a suitable solvent) may be pumped into a stirred holding tank or directly into the polymerization reactor.

In some variations, the liquid catalysts and/or catalyst precursors are dispensed from a shipping vessel/container into an intermediate, inert vessel to be mixed with suitable solvent, and then the catalyst solution is fed to the reactor or a pre-mix tank. The catalyst preparation system and the connections may be selected in such a way to ensure that the catalyst or precursors are not contacted by ambient atmosphere.

In some variations, the polymerization reactor is a PFR, the liquid catalyst (as a meld or as a solution in a suitable solvent) and BPL are fed to a small stirred tank and then the mixture is fed to the PFR. In other embodiments, the BPL and the liquid catalyst are fed to a pre-mixer installed at the inlet of the PFR. In yet another embodiment, the PFR has a static mixer, the reaction occurs on the shell side of the reactor, and the liquid catalyst and BPL are introduced at the inlet of the reactor and the static mixer elements mix the catalyst and BPL. In still another embodiment, the PFR has a static mixer, the reaction occurs on the shell side of the reactor, and the liquid catalyst is introduced into the PFR using metering pumps at multiple locations distributed along the lengths of the reactor.

In some embodiments, the homogeneous polymerization catalyst is delivered to the location of the polymerization reactor as a solid (for example, solid Al(TPP)Et or solid TBA acrylate), the solid catalyst is unpacked and loaded in hoppers under inert conditions (CO or inert gas), and the solids from hoppers are be metered into a suitable solvent before pumping into the polymerization reactors or mixing tanks.

Any suitable polymerization catalyst may be used in the polymerization process to convert the production stream entering the polymerization process to the PPL product stream. In some embodiments, the polymerization catalyst is heterogeneous with the polymerization reaction mixture. Any suitable heterogeneous polymerization catalyst capable of polymerizing BPL in the production stream to produce the PPL product stream may be used in the methods described herein.

In some embodiments, the heterogeneous polymerization catalyst comprises any of the homogeneous polymerization catalysts described above, supported on a heterogeneous support. Suitable heterogeneous supports may include, for example, amorphous supports, layered supports, or microporous supports, or any combination thereof. Suitable amorphous supports may include, for example, metal oxides (such as aluminas or silicas) or carbon, or any combination thereof. Suitable layered supports may include, for example, clays. Suitable microporous supports may include, for example, zeolites (such as molecular sieves) or cross-linked functionalized polymers. Other suitable supports may include, for example, glass surfaces, silica surfaces, plastic surfaces, metal surfaces including zeolites, surfaces containing a metallic or chemical coating, membranes (comprising, for example, nylon, polysulfone, silica), micro-beads (comprising, for example, latex, polystyrene, or other polymer), and porous polymer matrices (comprising, for example, polyacrylamide, polysaccharide, polymethacrylate).

In some embodiments, the heterogeneous polymerization catalyst is a solid-supported quaternary ammonium salt (for example, tetrabutylammonium (TBA) acrylate, TBA acetate, trimethylphenylammonium acrylate, or trimethylphenylammonium acetate) or a phosphine (for example, tetraphenyl phosphonium acrylate).

In some embodiments, the catalyst is solid-supported tetrabutylammonium acrylate, iron chloride, TBA acrylate, TBA acetate, trimethylphenylammonium acrylate, trimethylphenylammonium acetate, or tetraphenyl phosphonium acrylate.

In certain embodiments, conversion of the production stream entering the polymerization process to the PPL product stream utilizes a solid carboxylate catalyst and the conversion is conducted at least partially in the gas phase. In certain embodiments, the solid carboxylate catalyst in the polymerization process comprises a solid acrylic acid catalyst. In certain embodiments, the production stream enters the polymerization process as a liquid and contacted with a solid carboxylate catalyst to form the PPL product stream. In other embodiments, the production stream enters the polymerization process as a gas and contacted with a solid carboxylate catalyst to form the PPL product stream.

In some variations, the polymerization catalyst is a heterogeneous catalyst bed. Any suitable resin may be used for such a heterogeneous catalyst bed. In one embodiment, the polymerization catalyst is a heterogeneous catalyst bed packed in a tubular reactor. In some embodiments, the polymerization reactor system comprises a plurality of heterogeneous catalyst beds, wherein at least one catalyst bed is being used in the polymerization reactor, and at least one catalyst bed is not being used in the polymerization reactor at the same time. For example, the catalyst bed not actively being used may be being regenerated for later use, or may be stored as a back-up catalyst bed in case of catalyst failure of the actively used bed. In one embodiment, the polymerization reactor system comprises three heterogeneous catalyst beds, wherein one catalyst bed is being used in the polymerization reactor, one catalyst bed is being regenerated, and one catalyst bed is being stored as a back-up in case of catalyst failure.

In some variations, the heterogeneous polymerization catalyst is prepared in one location, then shipped to a second location where it is used in the polymerization reactor. In other embodiments, the heterogeneous polymerization catalyst is prepared at the location of the polymerization reactor (for example, to reduce exposure to moisture and/or oxygen).

In some embodiments, the polymerization process does not include solvent. In other embodiments, the polymerization process does include one or more solvents. Suitable solvents can include, but are not limited to: hydrocarbons, ethers, esters, ketones, nitriles, amides, sulfones, halogenated hydrocarbons, and the like. In certain embodiments, the solvent is selected such that the PPL product stream is soluble in the reaction medium.

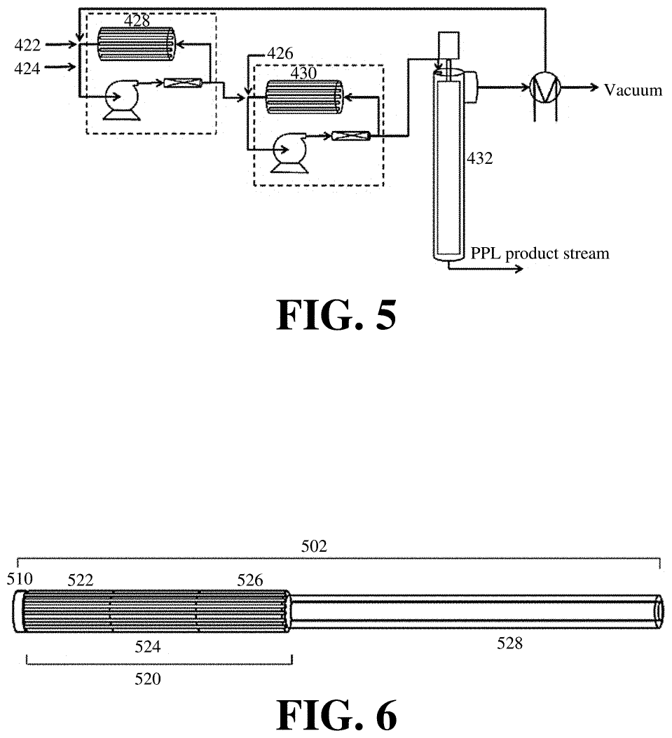

For example, with reference to polymerization process depicted in FIGS. 4 and 5, reactors 408 and/or 410 may be configured to receive solvent. For example, in one variation, polymerization process may further include a solvent source configured to feed solvent into reactors 408 and 410. In another variation, the BPL from production stream 402 may be combined with solvent to form the production stream containing BPL fed into reactor 408. In yet another variation, the polymerization catalyst from polymerization catalyst sources 404 and/or 406 may be combined with a solvent to form polymerization catalyst streams fed into the reactors.

The one or more polymerization reactors in the polymerization process may be any suitable polymerization reactors for the production of the PPL product stream from the production stream entering the polymerization process. For example, the polymerization reactor may be a CSTR, loop reactor, or plug flow reactor, or a combination thereof. In some embodiments, the polymerization process comprises a single reactor, while in other embodiments, the polymerization process comprises a plurality of reactors. In some variations, the BPL is completely converted to PPL in a polymerization reactor. In other variations, the BPL is not completely converted to PPL in a polymerization reactor, and the PPL stream exiting the polymerization reactor comprises unreacted BPL. In certain variations, the PPL stream comprising unreacted BPL is directed to a BPL/PPL separator to remove the BPL from the PPL. The BPL may then be recycled back into the polymerization reactor, as described, for example, in FIGS. 8, 9, 11 and 12 above.

In certain variations, the polymerization process comprises two reactors in series, wherein the purified BPL stream enters the first reactor and undergoes incomplete polymerization to produce a first polymerization stream comprising PPL and unreacted BPL, the first polymerization stream exits the outlet of the first reactor and enters the inlet of the second reactor to undergo additional polymerization. In some variations, the additional polymerization completely converts the BPL to PPL, and the PPL product stream exits the outlet of the second polymerization reactor.

In other variations, the additional polymerization incompletely converts the BPL to PPL, and the PPL product stream exiting the outlet of the second polymerization reactor comprises PPL and unreacted BPL. In certain variations, the PPL product stream enters a BPL/PPL separator to remove unreacted BPL from the PPL product stream. In certain variations, the unreacted BPL is recycled back into the polymerization process. For example, in some variations, the unreacted BPL is recycled to the first polymerization reactor or the second polymerization reactor, or both the first and the second polymerization reactors.

In some embodiments, the polymerization process comprises a series of one or more continuous CSTR reactors followed by a BPL/PPL separator (such as a wiped film evaporator (WFE) or distillation column). In other embodiments, the polymerization process comprises a series of one or more loop reactors followed by a BPL/PPL separator (such as a WFE or distillation column). In yet other embodiments, the polymerization process comprises a series of one or more in a series of one or more CSTR reactors followed by a polishing plug flow reactor (PFR) or by a BPL/PPL separator (Wiped Film Evaporator or Distillation column). In still other embodiments, the polymerization process comprises a series of one or more PFR optionally followed by a BPL/PPL separator (such as a WFE or distillation column).

In some embodiments, the polymerization process comprises greater than two polymerization reactors. For example, in certain embodiments, the polymerization process comprises three or more polymerization reactors, four or more polymerization reactors, five or more polymerization reactors, six or more polymerization reactors, seven or more polymerization reactors, or eight or more polymerization reactors. In some variations, the reactors are arranged in series, while in other variations, the reactors are arranged in parallel. In certain variations, some of the reactors are arranged in series while others are arranged in parallel.

FIGS. 4 and 5 depict exemplary PPL production systems comprising two polymerization reactors connected in series, and a PPL purification and BPL recycle system with a wiped film evaporator (WFE) for recycling of unreacted BPL back into the polymerization reactors. With reference to FIG. 4, the polymerization process includes BPL source 402 and polymerization catalyst source 404, configured to feed BPL and catalyst, respectively, into reactor 408. Reactor 408 includes a BPL inlet to receive BPL from the BPL source and a polymerization catalyst inlet to receive polymerization catalyst from the polymerization catalyst source. In some variations, the BPL inlet is configured to receive the BPL from the BPL source at a rate of 3100 kg/hr, and the first polymerization catalyst inlet is configured to receive the polymerization catalyst from the polymerization catalyst source at a rate of 0.1 to 5 kg/hr.

With reference again to FIG. 4, reactor 408 further includes a mixture outlet to output a mixture comprising PPL and unreacted BPL, to reactor 410. Reactor 410 is a second reactor positioned after reactor 408, and is configured to receive the mixture from reactor 408 and additional polymerization catalyst from polymerization catalyst source 406. In some variation, the mixture inlet of the second reactor is configured to receive the mixture from the first reactor at a rate of 4500 kg/hr, and the second polymerization catalyst inlet is configured to receive additional polymerization catalyst from the catalyst source at a rate of 0.1 to 4 kg/hr.

With reference again to FIG. 4, reactor 408 further includes a mixture outlet to output a mixture comprising PPL, and unreacted BPL to evaporator 412. In some variations, the mixture outlet is configured to output such mixture at a rate of 4500 kg/hr.

With reference to FIG. 5, the depicted polymerization process includes BPL source 422 and polymerization catalyst source 424, configured to feed BPL and catalyst, respectively, into reactor 428. Reactor 428 includes a BPL inlet to receive BPL from the BPL source and a polymerization catalyst inlet to receive polymerization catalyst from the polymerization catalyst source. In some variations, the BPL inlet is configured to receive the BPL from the BPL source at a rate of 3100 kg/hr, and the first catalyst inlet is configured to receive the catalyst from the catalyst source at a rate of 0.1 to 5 kg/hr.

With reference again to FIG. 5, reactor 428 further includes a mixture outlet to output a mixture comprising PPL and unreacted BPL to reactor 430. Reactor 430 is a second reactor positioned after reactor 428, and is configured to receive the mixture from reactor 428 and additional polymerization catalyst from polymerization catalyst source 426. In some variation, the mixture inlet of the second reactor is configured to receive the mixture from the first reactor at a rate of 4500 kg/hr, and the second polymerization catalyst inlet is configured to receive additional polymerization catalyst from the polymerization catalyst source at a rate of 0.1 to 4 kg/hr.

In some variations, the mixture output from reactor 410 (FIG. 4) and reactor 430 (FIG. 5) is made up of at least 95% wt PPL.

Such mixture may be output from the second reactor to an evaporator. Evaporator 412 (FIG. 4) and 432 (FIG. 5) may be, for example, a wiped film evaporator, thin film evaporator, or falling film evaporator. The evaporator is configured to produce a PPL product stream.

In some variations, the evaporator is configured to produce a PPL product stream having a purity of at least 98%, at least 98.5%, or at least 99%. In other variations, the evaporator is configured to produce a PPL product stream having less 0.1% wt of BPL.

In some variations, the polymerization process further includes one or more heat exchangers. With reference to FIG. 4, BPL from BPL source 402 may be passed through heat exchanger before such BPL stream is fed into reactor 408.

It should generally be understood that the polymerization is an exothermic reaction. Thus, in other variations, reactors 408 and 410 (FIG. 4) may further include a connection to at least one heat exchanger. With reference to FIG. 5, reactors 428 and 430 (FIG. 5) may further include a connection to at least one heat exchanger.

In some variations, the first reactor in the polymerization process may be configured to remove heat produced at a rate of 1.8×10⁹ J/hr. In some variations, the second reactor may be configured to remove heat produced at a rate of 1.8×10⁹ J/hr. In other variations, the heat from the first reactor and heat from the second reactor are removed at a ratio between 0.25 and 4.

The reactors of polymerization process may include any suitable reactors, including, for example, continuous reactors or semi-batch reactors. In one variation, with reference to FIG. 4, the reactors may be continuous-flow stirred-tank reactors. The reactors may also include the same or different stirring devices. For example, in one variation, reactor 408 may include a low velocity impeller, such as a flat blade. In other variation, reactor 410 may include a low shear mixer, such as a curved blade.

A skilled artisan would recognize that the choice for the mixing device in each of the reactors may depend on various factors, including the viscosity of the mixture in the reactor. For example, the mixture in the first reactor may have a viscosity of 1000 cP. If the viscosity is 1000 cP, then a low velocity impeller may be desired. In another example, the mixture in the second reactor may have a viscosity of 5000 cP. If the viscosity is 5000 cP, then a low shear mixer may be desired.

In another variation, with reference to FIG. 5, the reactors may be loop reactors.

It should be understood that while FIGS. 4 and 5 depict the use of two reactors configured in series, other configurations are considered. For example, in other exemplary variations of the polymerization process, three reactors may be employed. In yet other variations where a plurality of reactors is used in the polymerization process, they may be arranged in series or in parallel.

FIG. 6 depicts yet another exemplary polymerization process, which includes a BPL polymerization reactor. The polymerization reactor includes mixing zone 510 configured to mix the production stream entering the polymerization process and catalyst, and a plurality of cooling zones 520 positioned after the mixing zone. The polymerization reactor has reaction length 502, wherein up to 95% of the BPL in the entering production stream is polymerized in the presence of the catalyst to form PPL in the first 25% of the reaction length. In some variations of the system depicted in FIG. 6, the BPL is completely converted to PPL. Such a system may be used, for example, in the complete conversion of BPL to PPL as described above for FIGS. 7, 10, 13 and 14.

In some variations of a polymerization reactor, the plurality of cooling zones includes at least two cooling zones. In one variation, the plurality of cooling zones includes two cooling zones or three cooling zones.

For example, polymerization reactor 500 as depicted in FIG. 6 has three cooling zones 522, 524 and 526. In one variation, the three cooling zones are connected serially in the first 25% of the reaction length. In another variation, cooling zone 522 is configured to receive a mixture of BPL and the catalyst from the mixing zone at a rate of 3100 kg/hr; cooling zone 524 is configured to receive a mixture of the BPL, the catalyst and PPL produced in cooling zone 522 at a rate of 3100 kg/hr; and cooling zone 526 is configured to receive a mixture of the BPL, the catalyst, the PPL produced in cooling zone 522, and PPL produced in cooling zone 524 at a rate of 3100 kg/hr.

In certain embodiments, the first 25% of the reaction length is a shell and a tube heat exchanger. In one variation, the shell may be configured to circulate a heat transfer fluid to maintain a constant temperature in reaction length 502. In another variation, the tube heat exchanger is configured to remove heat produced in the first reaction zone.

With reference again to FIG. 6, polymerization reactor 500 further includes end conversion zone 528 connected to plurality of cooling zones 520. In some variations, the end conversion zone is configured to receive a mixture of the BPL, the catalyst, and the PPL produced in plurality of cooling zones at a rate of 3100 kg/hr. In one variation, the end conversion zone has no cooling load.

In one variation, the polymerization reactor is a plug flow reactor or a shell-and-tube reactor.

The one or more polymerization reactors used in the methods described herein may be constructed of any suitable material compatible with the polymerization. For example, the polymerization reactor may be constructed from stainless steel or high nickel alloys, or a combination thereof.

In some embodiments, the polymerization process comprises a plurality of polymerization reactors, and the polymerization catalyst is introduced only into the first reactor in the series. In other embodiments, the polymerization catalyst is added separately to each of the reactors in the series. For example, referring again to FIG. 4, depicted is a polymerization process comprising two CSTR in series, wherein polymerization catalyst is introduced to the first CSTR, and polymerization catalyst is separately introduced to the second CSTR. In other embodiments, a single plug flow reactor (PFR) is used, and polymerization catalyst is introduced at the beginning of the reactor, while in other embodiments polymerization catalyst is introduced separately at a plurality of locations along the length of the PFR. In other embodiments, a plurality of PFR is used, and polymerization catalyst is introduced at the beginning of the first PRF. In other embodiments, polymerization catalyst is introduced at the beginning of each PFR used, while in still other embodiments polymerization catalyst is introduced separately at a plurality of locations along the length of each PFR.

The polymerization reactor may comprise any suitable mixing device to mix the polymerization reaction mixture. Suitable mixing devices may include, for example, axial mixers, radial mixers, helical blades, high-shear mixers, or static mixers. Suitable mixing devices may comprise single or multiple blades, and may be top, bottom, or side mounted. The polymerization reactor may comprise a single mixing device, or multiple mixing devices. In some embodiments, a plurality of polymerization reactors is used, and each polymerization reactor comprises the same type of mixing device. In other embodiments, each polymerization reactor comprises a different type of mixing device. In yet other embodiments, some polymerization reactors comprise the same mixing device, while others comprise different mixing devices.

In some embodiments, the production system described herein further comprises a PPL stream processing system configured to receive the PPL product stream and produce solid PPL. For example, in one embodiment, the PPL product stream is fed into at least one inlet of a PPL stream processing system, and solid PPL exits at least one outlet of the PPL stream processing system. The PPL stream processing system may be configured to produce solid PPL in any suitable form. For example, in some embodiments, the PPL stream processing system is configured to produce solid PPL in pelleted form, flaked form, granulated form, or extruded form, or any combinations thereof. Thus, solid PPL flakes, solid PPL pellets, solid PPL granules, or solid PPL extrudate, or any combinations thereof, may exit the outlet of the PPL stream processing system. The PPL stream processing system may include one or more flaking devices, pelleting devices, extrusion devices, or granulation devices, or any combinations thereof.

In certain embodiments, the production system described herein produces a PPL product stream at a first location, the PPL product stream is processed to produce solid PPL, and the solid PPL is converted to an AA product stream in a second location. In some embodiments, the first location and the second location are at least 100 miles apart. In certain embodiments, the first location and the second location are between 100 and 12,000 miles apart. In certain embodiments, the first location and the second location are at least 250 miles, at least 500 miles, at least 1,000 miles, at least 2,000 or at least 3,000 miles apart. In certain embodiments, the first location and the second location are between about 250 and about 1,000 miles apart, between about 500 and about 2,000 miles apart, between about 2,000 and about 5,000 miles apart, or between about 5,000 and about 10,000 miles apart. In certain embodiments, the first location and the second location are in different countries. In certain embodiments, the first location and the second location are on different continents.

In certain embodiments, the solid PPL is transported from the first location to the second location. In some embodiments, the solid PPL is transported a distance of more than 100 miles, more than 500 miles, more than 1,000 miles, more than 2,000 miles or more than 5,000 miles. In certain embodiments, the solid PPL is transported a distance of between 100 and 12,000 miles, between about 250 and about 1000 miles, between about 500 and about 2,000 miles, between about 2,000 and about 5,000 miles, or between about 5,000 and about 10,000 miles. In some embodiments, the solid PPL is transported from a first country to a second country. In certain embodiments, the solid PPL is transported from a first continent to a second continent.

In certain embodiments, the solid PPL is transported from the North America to Europe. In certain embodiments, the solid PPL is transported from the North America to Asia. In certain embodiments, the solid PPL is transported from the US to Europe. In certain embodiments, the solid PPL is transported from the US to Asia. In certain embodiments, the solid PPL is transported from the Middle East to Asia. In certain embodiments, the solid PPL is transported from the Middle East to Europe. In certain embodiments, the solid PPL is transported from Saudi Arabia to Asia. In certain embodiments, the solid PPL is transported from Saudi Arabia to Europe.

The solid PPL may be transported by any suitable means, including, for example, by truck, train, tanker, barge, or ship, or any combinations of these. In some embodiments, the solid PPL is transported by at least two methods selected from truck, train, tanker, barge, and ship. In other embodiments, the solid PPL is transported by at least three methods selected from truck, train, tanker, barge, and ship.

In some embodiments, the solid PPL is in the form of pellets, flakes, granules, or extrudate, or any combination thereof. In some variations, the solid PPL is converted to an AA product stream using the thermolysis reactor as described herein. In some variations, the solid PPL is fed into an inlet of the thermolysis reactor and is converted to an AA product stream. In other embodiments, the solid PPL is converted to molten PPL, and the molten PPL is fed into an inlet of the thermolysis reactor as described herein and converted to an AA product stream.

Acrylic Acid Production System

Polypropiolactone (PPL) can generally be converted to acrylic acid (AA) according to the following scheme:

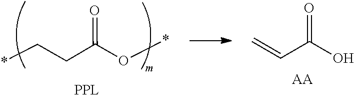

In certain embodiments, the polypropiolactone produced undergoes thermolysis continuously (e.g. in a fed batch reactor or other continuous flow reactor format). In certain embodiments, the continuous thermolysis process is linked to a continuous polymerization process to provide acrylic acid at a rate matched to the consumption rate of the reactor.

In some embodiments, the thermolysis reactor is a fluidized bed reactor. Inert gas may be used to fluidize inert solid heat transfer medium (HTM), and polypropiolactone is fed to the reactor. In some variations, the polypropiolactone may be fed to the reactor in molten form, for example, via a spay nozzle. The molten form may help facilitate the dispersion of polypropiolactone inside the reactor.

The reactor may be equipped with a cyclone that returns HTM solid back to the reactor. The inert gas, acrylic acid, and higher boiling impurities (such as succinic anhydride and acrylic acid dimer) are fed from the cyclone to a partial condenser where impurities are separated. For example, the condenser may be used to condense the high boiling impurities, and such impurities can then be purged from the reactor as a residual waste stream.

Acrylic acid with the inert gas may be fed to a second condenser where the acrylic acid and the inert gas are separated. A liquid acrylic acid stream is output from the second condenser, and the inert gas is output as a separate stream that may be returned back to the reactor to fluidize the heat transfer solid. The acrylic acid stream may be used for condensation/absorption and then storage.

The residual waste stream purged from the reactor may include, for example, high boiling organics (or organic heavies), for example, resulting from the polymerization catalyst and succinic anhydride. In some embodiments, the high boiling organics (or organic heavies) may include any compounds which are not acrylic acid. In certain embodiments, the high boiling organics (or organic heavies) may include any compounds which remain in the bottoms stream after condensing the acrylic acid in the acrylic acid production system. In some embodiments, the high boiling organics (or organic heavies) may include succinic anhydride or polymerization catalyst. In some embodiments, the high boiling organics (or organic heavies) have a boiling point higher than acrylic acid.

In other embodiments, the thermolysis reactor is a moving bed reactor. Polypropiolactone is fed into a moving bed reactor as a solid and acrylic acid exits the reactor as a vapor stream and is then condensed.

In some variations, the thermolysis process is operated under an oxygen and water free atmosphere. For example, in certain variations, the amount of oxygen present in the thermolysis reactor is less than 1 wt %, less than 0.5 wt %, less than 0.01 wt %, or less than 0.001 wt %. In certain variations, the amount of water present in the thermolysis reactor is less than 1 wt %, less than 0.5 wt %, less than 0.01 wt %, or less than 0.001 wt %.

In some variations, acrylic acid produced according to the systems and methods described herein has a purity of at least 98%, at least 98.5%, at least 99%, at least 99.1%, at least 99.2%, at least 99.3%, at least 99.4%, at least 99.5%, at least 99.6%, at least 99.7%, at least 99.8%, or at least 99.9%; or between 99% and 99.95%, between 99.5% and 99.95%, between 99.6% and 99.95%, between 99.7% and 99.95%, or between 99.8% and 99.95%.

In other variations, acrylic acid produced according to the systems and methods described herein is suitable to make high molecular weight polyacrylic acid. In certain variations, acrylic acid produced according to the systems and methods described herein may have a lower purity, such as 95%. Thus, in one variation, the acrylic acid has a purity of at least 95%.

In yet other variations, the acrylic acid has:
(i) a cobalt level of less than 10 ppm, less than 100 ppm, less than 500 ppm, less than 1 ppb, less than 10 ppb, or less than 100 ppb; or
(ii) an aluminum level of less than 10 ppm, less than 100 ppm, less than 500 ppm, less than 1 ppb, less than 10 ppb, or less than 100 ppb; or
(iii) a beta-propiolactone level of less than 1 ppm, less than 10 ppm, less than 100 ppm, less than 500 ppm, less than 1 ppb, or less than 10 ppb;
(iv) an acrylic acid dimer level of less than 2000 ppm, less than 2500 ppm, or less than 5000 ppm; or
(v) a water content of less than 10 ppm, less than 20 ppm, less than 50 ppm, or less than 100 ppm,
or any combination of (i) to (v).

Unlike known methods to produce acrylic acid, acetic acid, furfurals and other furans are not produced and thus, are not present in the acrylic acid produced.

Acrylic acid may be used to make polyacrylic acid for superabsorbent polymers (SAPs) in disposable diapers, training pants, adult incontinence undergarments and sanitary napkins. The low levels of impurities present in the acrylic acid produced according to the systems and methods herein help to facilitate a high-degree of polymerization to acrylic acid polymers (PAA) and avoid adverse effects from by-products in end applications. For example, aldehyde impurities in acrylic acid hinder polymerization and may discolor the polymerized acrylic acid. Maleic anhydride impurities form undesirable copolymers which may be detrimental to polymer properties. Carboxylic acids, e.g., saturated carboxylic acids that do not participate in the polymerization, can affect the final odor of PAA or SAP-containing products and/or detract from their use. For example, foul odors may emanate from SAP that contains acetic acid or propionic acid and skin irritation may result from SAP that contains formic acid. The reduction or removal of impurities from petroleum-based acrylic acid can be costly, whether to produce petroleum-based crude acrylic acid or petroleum-based acrylic acid.

### EXAMPLES

The following Examples are merely illustrative and are not meant to limit any aspects of the present disclosure in any way.

### Example 1

This example describes an exemplary protocol for producing an exemplary heterogeneous catalyst (5).

With reference to FIG. 18A, meso-tetraphenylporphyrin (TPP) (1) undergoes sulfonation in the presence of concentrated sulfuric acid to yield meso-tetra(4-sulfonatophenyl)porphyrin (2). TPP suspended in sulfuric acid is heated via a steam bath for 6 h. Water is then added to the reaction mixture and the protonated porphyrin is collected by filtration. Neutralization by sodium bicarbonate is carried out in a mixture of water and Celite with the porphyrin. Filtration is used to remove the Celite and unreacted TPP. Further purification may be employed to remove inorganic contaminants.

With reference to FIG. 18B, meso-tetra(4-sulfonatophenyl)porphyrin (2) is then metallated with a Lewis acid to yield metallated TPP (3).

With reference to FIG. 18C, reaction of metallated sulfonatophenyl porphyrin (3) with NaCo(CO)₄ yields a sulfonate functionalized Lewis acid-Co(CO)₄ catalyst (4).

With reference to FIG. 18D, sulfonatophenyl porphyrin (4) undergoes heterogenization by grafting the sulfonate groups onto activated silica as a support in anhydrous dichloromethane to yield catalyst (5).

### Example 2

This example describes an exemplary protocol to covalently tether porphyrin or salen ligands to support structures via reaction of chloro functionalized porphyrin or salen ligands.

With reference to FIG. 19A, meso-tetra(4-chlorophenyl)porphyrin (1) is tethered by the reaction of the meso-tetra(4-chlorophenyl)porphyrin with aminopropyl functionalized grafted siloxanes to the support attaches the porphyrin to the support through an amine linkage. The synthesis of the aminopropyl functionalized solid support is achieved by the reaction of 3-aminopropyltriethoxysilane with silanol groups on the surface of the support that leads to anchoring through a condensation reaction.

With reference to FIG. 19B the tethered porphyrin is metallated with a Lewis acid (such as AlEt₃ or (Et)₂AlCl as depicted in the figure) to yield a porphyrin (3) that has been metallated and tethered.

With reference to FIG. 19C, reaction of Lewis acid metallated chlorophenyl porphyrin (2) with Co₂(CO)₈ or NaCo(CO)₄ yields a tethered meso-tetra(4-chlorophenyl)porphyrin Co(CO)₄ (4).

The chloro functionalities are used as attachment points for tethering of the porphyrin structure to a support, such as silica or zeolite, for heterogenization of the catalyst. With reference to FIG. 19A,

### Example 3

This example describes an exemplary protocol to encapsulate salen ligands within the pores of zeolites, including microporous zeolites (referred to as a “ship-in-a-bottle” catalyst). The encapsulation procedure includes delumination of the zeolite, followed by ion exchange with a cationic metal (M). The ligand that surrounds the cationic metal is then synthesized first by reaction of the zeolite with a diamine, and then further reaction with an aldehyde. FIG. 20 depicts encapsulated metallated salen ligand within a zeolite pore. A source of cobalt is then introduced, such as in the form of Co₂(CO)₈.

The exemplary procedure above can also generally be applied to porphyrin ligands, provided that the zeolite has the appropriate pore size to encapsulate the porphyrin ligands.

1. A compound, comprising:
a solid support;
at least one ligand coordinated to a metal atom to form a metal complex;
at least one anionic metal carbonyl moiety coordinated to the metal complex; and
at least one linker moiety covalently tethering the ligand to the solid support.

2. The compound of claim 1, wherein the at least one ligand is a porphyrin ligand or a salen ligand.

3. The compound of claim 1, wherein the at least one anionic metal carbonyl moiety is a cobalt carbonyl moiety.

4. The compound of claim 2, wherein the at least one anionic metal carbonyl moiety is [Co(CO)₄]⁻.

5. The compound of claim 1, wherein the at least one linker moiety comprises a sulfonate moiety or an aminosiloxane moiety.

6. (canceled)

7. The compound of claim 1, wherein the solid support comprises silica/alumina, pyrogenic silica, alumina, carbon, clay, silica microbeads, magnesia, titania, zirconia, zincate, or microporous zeolite, or any combination thereof.

8. The compound of claim 1, wherein the solid support comprises silica, wherein the silica has a surface comprising silanols.

9. The compound of claim 1, wherein the at least one linker moiety comprises a sulfonate moiety.

10. The compound of claim 9, wherein the solid support comprises silica, and wherein the silica has a surface comprising silanols, and wherein the at least one linker moiety coordinates with at least a portion of the silanols on the surface of the silica.

11. The compound of claim 1, wherein the at least one linker moiety comprises an aminosiloxane moiety.

12. The compound of claim 11, wherein the solid support comprises silica or zeolite, and wherein the aminosiloxane moiety comprises (i) an amino group connected to the ligand, and (ii) a siloxane group connected to the solid support.

13. (canceled)

14. (canceled)

15. The compound of claim 1, wherein the metal complex has a structure of formula (M-A1): 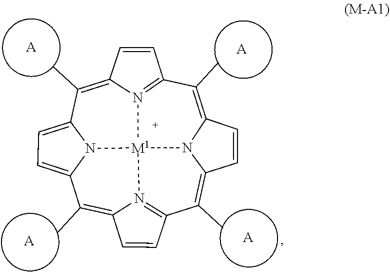
wherein:
M¹ is a metal atom, and
each ring A is independently optionally substituted, and wherein at least one ring A is connected by the linker moiety to the solid support.

16. The compound of claim 15, wherein each ring A is independently a 6 membered carbocyclic moiety, or a 6-membered heterocyclic moiety.

17. (canceled)

18. (canceled)

19. The compound of claim 16, wherein the 6-membered heterocyclic moiety comprises at least one nitrogen atom.

20. The compound of claim 1, wherein the metal complex has a structure of formula (M-B): 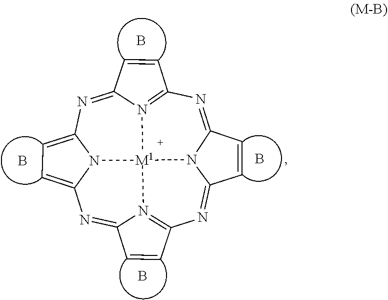
wherein:
M¹ is a metal atom, and
each ring B is independently optionally substituted, and wherein at least one ring B is connected by the linker moiety to the solid support.

21. The compound of claim 20, wherein each ring B is independently a 6-membered carbocyclic moiety, or a 6-membered heterocyclic moiety.

22. (canceled)

23. (canceled)

24. The compound of claim 21, wherein the 6-membered heterocyclic moiety comprises at least one nitrogen atom.

25. The compound of claim 1, wherein the metal complex has a structure of formula (M-C1): 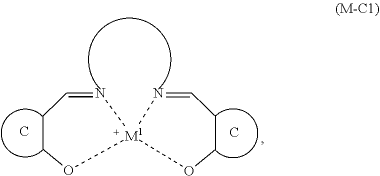
wherein:
M¹ is a metal atom, [symbol] is an optionally substituted moiety linking the two nitrogen atoms of the diamine portion of the ligand, and
each ring C is independently optionally substituted, and wherein at least one ring C is connected by the linker moiety to the solid support.

26. The compound of claim 25, wherein each ring C is independently a 6-membered carbocyclic moiety, or a 6-membered heterocyclic moiety.

27. (canceled)

28. (canceled)

29. The compound of claim 26, wherein the 6-membered heterocyclic moiety comprises at least one nitrogen atom.

30. The compound of claim 1, wherein the metal atom is Zn, Cu, Mn, Co, Ru, Fe, Rh, Ni, Pd, Mg, Al, Cr, Ti, Fe, In, or Ga.

31.-37. (canceled)
